SEQUENCE LISTING (15 sequences)

SEQ ID NO: 1 - protein, 108 aa, Artificial Sequence
  DCR-2 VH region
MESGGGLVQPGGPLKLSCAASGFGFSGSWMSWVRQAPGKGLEWIGQINPDSSTINYTPSLKDKFIISRDNAKNTLYLQINKVRSEDTALYYCARRGFFEGYSAWFAYW

SEQ ID NO: 2 - protein, 8 aa, Artificial Sequence
  CDR1 of VH of DCR-2
GFGFSGSW

SEQ ID NO: 3 - protein, 8 aa, Artificial Sequence
  CDR2 of VH of DCR-2
INPDSSTI

SEQ ID NO: 4 - protein, 15 aa, Artificial Sequence
  CDR3 of VH of DCR-2
ARRGFFEGYSAWFAY

SEQ ID NO: 5 - protein, 100 aa, Artificial Sequence
  VL of DCR-2
ILMTQTPKFLLVSAGDRVTITCKASQSVSNDVAWYQQKPGQSPSLLIYYASNRNTGVPDRFTGSGYETDFTFTISTVQAEDLAVYFCQQDYTSPWTFGGG

SEQ ID NO: 6 - protein, 6 aa, Artificial Sequence
  CDR1 of VL of DCR-2
QSVSND

SEQ ID NO: 7 - protein, 3 aa, Artificial Sequence
  CDR2 of VL of DCR-2
YAS

SEQ ID NO: 8 - protein, 9 aa, Artificial Sequence
  CDR3 of VL of DCR-2
QQDYTSPWT

SEQ ID NO: 9 - protein, 324 aa, Artificial Sequence
  VH region of DCR2
ATGGAGTCTGGAGGTGGCCTGGTGCAGCCTGGAGGACCCCTGAAACTCTCCTGTGCAGCCTCAGGATTCGGTTTTAGTGGATCTTGGATGAGTTGGGTCCGGCAGGCTCCAGGGAAAGGGCTAGAATGGATTGGACAAATTAATCCAGATAGCAGTACGATAAATTATACACCATCTCTAAAGGATAAATTCATCATCTCCAGAGACAACGCCAAAAATACCCTGTACCTGCAAATTAACAAAGTGAGATCTGAGGACACAGCCCTTTATTACTGTGCAAGACGGGGGTTCTTTGAAGGTTACTCCGCCTGGTTTGCTTACTGG

SEQ ID NO: 10 - DNA, 300 bp, Artificial Sequence
  VL region of DCR2
attttgatgacccagactcccaaattcctgcttgtatcagcaggagacagggtgaccataacctgcaaggccagtcagagtgtgagtaatgatgtagcttggtaccaacagaagccagggcagtctccttcactcctgatatactatgcatccaatcgcaacactggagtccctgatcgcttcactggcagtggatatgagacggatttcactttcaccatcagcactgtgcaggctgaagacctggcagtttatttctgtcagcaggattatacctctccgtggacgttcggtggaggc

SEQ ID NO: 11 - DNA, 1422 bp, Artificial Sequence
  codon optimised chimeric heavy chain
atggtcctgagcctgctgtacctgctgacagctctgcctggcatcctgtctgaggtccagctgcaagagtctggccccatggaatctggcggaggattggttcaacctggcggccctctgaagctgtcttgtgccgcttctggcttcggcttctccggctcttggatgtcctgggtccgacaggctcctggcaaaggcctggaatggatcggccagatcaaccccgactcctccaccatcaactacacccctagcctgaaggacaagttcatcatctcccgggacaacgccaagaacaccctgtacttgcagatcaacaaagtgcggagcgaggacaccgctctgtactactgtgccagacggggcttcttcgagggctactctgcttggtttgcctactggggccagggcacactggtcacagtttctgccgcctctaccaagggacccagcgttttccctctggctccatcctccaagtctacctctggcggaacagctgctctgggctgcctggtcaaggactactttcctgagccagtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaatgccaagaccaagcctagagaggaacagtacaactccacctatagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaag

SEQ ID NO: 12 - DNA, 702 bp, Artificial Sequence
  codon optimised chimeric light chain
atggactctcaggcccaggtgctgatgctgctgctgttgtgggtgtccggcacctgtggcgacatcctgatgacccagactcctaagttcctgctggtgtctgccggcgacagagtgaccatcacatgcaaggcctctcagtccgtgtccaacgacgtggcctggtatcagcagaagcctggccagtctcctagcctgctgatctactacgcctccaacagaaacaccggcgtgcccgatagattcaccggctctggctacgagacagacttcaccttcaccatctccaccgtgcaggccgaggatctggccgtgtacttctgccagcaagactacacctctccatggacctttggcggaggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgc

SEQ ID NO: 13 - protein, 473 aa, Artificial Sequence
  heavy chain encoded by optimised nucleotide sequence
MVLSLLYLLTALPGILSEVQLGESGPMESGGGLVQPGGPLKLSCAASGFGFSGSWMSWVRQAPGKGLEWIGQINPDSSTINYTPSLKDKFIISRDNAKNTLYLQINKVRSEDTALYYCARRGFFEGYSAWFAYWGGGGTLVTVSAASTKGPSVFPLAPSSKSTSGGTAALGCLVKDYFPEPVTVSWNSGALTSGVHTFPAVLGSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSVMHEALHNHYTQKSLSLSPGK

SEQ ID NO: 14 - protein, 234 aa, Artificial Sequence
  light chain encoded by codon optimised nucleotide sequence
MDSQAQVLMLLLLWVSGTCGDILMTQTPKFLLVSADGRVTITCKASQSVSNDVAWYQQKPGQSPSLLIYYASNRNTGVPDRFTGSGYETDFTFTISTVQAEDLAVYFCQQDYTSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 108 aa, Artificial Sequence; identical to SEQ ID NO: 1
  humanised VH of DCR-2

(19) United States
(12) Patent Application Publication
(10) Pub. No.: US 2022/0296727 A1
(43) Pub. Date: Sep. 22, 2022

(54) Immunoconjugate

(71) Applicant: DendroCyte BioTech Pty Ltd, Balmain, New South Wales (AU)

(72) Inventors: Georgina Jane CLARK, Balmain (AU); Edward ABADIR, Camperdown (AU)

(73) Assignee: DendroCyte BioTech Pty Ltd, Balmain, New South Wales (AU)

(21) Appl. No.: 17/631,259

(22) PCT Filed: Jul. 30, 2020
(86) PCT No.: PCT/AU2020/050781

§ 371 Date: Jan. 28, 2022

(30) Foreign Application Priority Data
Jul. 30, 2019 (AU) 2019902714

(51) Int. Cl. A61K 47/68; A61K 31/5517; A61K 31/7076; C07K 16/28; A61P 43/00

(52) U.S. Cl. CPC A61K 47/6803; A61K 47/6849; A61K 31/5517; A61K 31/7076; C07K 16/2803; A61P 43/00; A61K 2039/505

(57) ABSTRACT

The present disclosure relates to an immunoconjugate including an antibody, or antigen binding fragment thereof, that specifically binds to CD300f, linked to at least one cytotoxic agent, and to compositions including the immunoconjugate, and use of the immunoconjugate for depleting haematopoietic stem and progenitor cells, for preparing a subject haematopoietic stem cell transplantation, and for the treatment of CD300f associated conditions.

### TECHNICAL FIELD

The present disclosure relates to an immunoconjugate that specifically binds to a CD300f polypeptide, compositions comprising the immunoconjugate, and to the use of the immunoconjugate for depleting haematopoietic stem and progenitor cells, for preparing a subject for haematopoietic stem cell transplantation, and for the treatment of CD300f associated conditions.

### BACKGROUND

Haematological diseases are disorders of the blood and blood-forming organs. Haematological diseases include haematological genetic disorders, such as congenital metabolic defects, hemoglobinopathies, and myelodysplastic and myeloproliferative syndromes, and haematological malignancies, such as lymphomas, and leukemias, such as Acute Myeloid Leukemia (AML).

AML is a cancer of the myeloid line of blood cells. AML is characterized by rapid growth of abnormal white blood cells that accumulate in the bone marrow and interfere with production of normal blood cells. AML is the most common acute leukemia affecting adults, and the incidence of the AML increases with age. As a consequence, AML is expected to increase in incidence as the population ages.

Despite major advances in understanding the pathogenesis of haematological diseases, patient outcomes remain unsatisfactory. A treatment option for haematological diseases and conditions is a haematological stem cell transplant, typically in combination with chemotherapy or radiotherapy.

While haematopoietic stem cell transplantation can be used in the treatment of myeloid leukaemias (e.g., AML) and other haematological malignancies and genetic disorders, substantial donor engraftment is only possible when the haematopoietic stem cell bone marrow niche of the patient has been cleared of recipient cells to allow space for donor cells to reside. To achieve complete engraftment, conditioning regimens are employed to deplete the recipient haematopoietic stem cell bone marrow niche, to suppress anti-graft immune response, and to reduce the burden of residual disease. Conditioning regimens traditionally incorporate radiation and/or chemotherapeutic agents. Alkylating agents such as busulfan, melphalan or irradiation deplete haematopoietic stem and progenitor cells (HSPC) while lymphodepleting agents such as fludarabine and cyclophosphamide deplete recipient lymphocytes. However, radiation and chemotherapeutic agents, such as alkylating agents, can be too harsh for certain patient cohorts. For example, aged patients, or patients suffering from haematological genetic disorders, are often unsuitable for receiving treatment with radiation and chemotherapeutic agents to deplete their haematopoietic stem and progenitor cells. As a consequence, use of effective conditioning regimens using radiation or chemotherapeutic agents including alkylating agents are not always possible for treatment of haematological malignancies, and are rarely possible for treatment of haematological genetic disorders. This is a problem for haematological malignancies, such as AML, as older patients constitute the majority of those diagnosed with AML.

Despite advances in haematopoietic stem cell transplantation, treatment related mortality (TRM) remains significant, especially in those older than 65 years (Kroger N., Blood. 2012; 119(24):5632-5639).

What is needed are alternative molecules which bind targets on haematopoietic stem cells, progenitor cells and haematological malignancies for treatment of haematological malignancies such as myeloid leukaemias, and for use in haematopoietic stem cell transplants.

### SUMMARY

CD300f is a member of the CD300f family of immunoregulatory molecules encoded by a gene complex on chromosome 17q25. It is a transmembrane glycoprotein with a cytoplasmic region and an extracellular domain. The cytoplasmic region contains both inhibitory ITIMs and PI3K phosphorylation site motifs. CD300f is expressed on healthy myeloid cells, including antigen presenting cells. CD300f is expressed on a high proportion of haematopoietic stem and progenitor cells, and in haematological malignancies, such as AML. CD300f is therefore a target for treatment of conditions associated with expression of CD300f, such as AML, and for depletion of haematopoietic stem cells and progenitor cells, such as in haematopoietic stem cell transplantation.

DCR-2 is a monoclonal antibody which specifically binds to the extracellular domain of CD300f. The inventors have found that DCR-2 is effective in killing leukemic cells when coupled with a pyrrolobenzodiazepine (PBD) moiety.

A first aspect provides an immunoconjugate comprising an antibody, or antigen binding fragment thereof, linked to at least one pyrrolobenzodiazepine moiety, wherein the antibody, or antigen binding fragment thereof, specifically binds to an extracellular domain of CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

A second aspect provides an immunoconjugate comprising an antibody, or antigen binding fragment thereof, linked to at least one pyrrolobenzodiazepine moiety, wherein the antibody, or antigen binding fragment thereof, specifically binds to an extracellular domain of CD300f, and comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

A third aspect provides an immunoconjugate comprising an antibody, or antigen binding fragment thereof, linked to at least one pyrrolobenzodiazepine moiety, wherein the antibody, or antigen binding fragment thereof, specifically binds to an extracellular domain of CD300f, and comprises a heavy chain variable region which comprises the amino acid sequence represented by SEQ ID NO: 1, and/or a light chain variable region which comprises the amino acid sequence represented by SEQ ID NO: 5.

A fourth aspect provides a composition comprising the immunoconjugate of any one of the first to third aspects.

A fifth aspect provides a method of treating a condition associated with CD300f expression in a subject in need thereof, comprising administering to the subject an effective amount of an immunoconjugate of any one of the first to third aspects, or a composition of the fourth aspect.

An alternative fifth aspect provides an immunoconjugate of any one of the first to third aspect, or a composition of the fourth aspect, for use in treating a condition associated with CD300f expression in a subject in need thereof; or use of an immunoconjugate of any one of the first to third aspects, or a composition of the fourth aspect, in the manufacture of a medicament for treating a condition associated with CD300f expression in a subject in need thereof.

A sixth aspect provides a kit comprising an immunoconjugate of any one of the first to third aspects, or a composition of the fourth aspect.

A seventh aspect provides a method of preparing a subject for haematopoietic stem cell transplantation, comprising administering to the subject:
(a) an effective amount of an immunoconjugate comprising an antibody, or an antigen binding fragment thereof, linked to at least one cytotoxic agent, wherein the antibody, or antigen binding fragment thereof, specifically binds CD300f; and
(b) an effective amount of a lymphodepleting agent.

An alternative seventh aspect provides:
(a) an immunoconjugate comprising an antibody, or an antigen binding fragment thereof, linked to at least one cytotoxic agent, wherein the antibody, or antigen binding fragment thereof, specifically binds an extracellular domain of CD300f; and
(b) a lymphodepleting agent,
   for use in preparing a subject for haematopoietic stem cell transplantation; or use of an immunoconjugate comprising an antibody, or an antigen binding fragment thereof, linked to at least one cytotoxic agent, wherein the antibody, or antigen binding fragment thereof, specifically binds CD300f, in the manufacture of a medicament for preparing a subject for haematopoietic stem cell transplantation, wherein the subject is being, has been, or is to be, administered a lymphodepleting agent.

An eighth aspect provides a method of preparing a subject for haematopoietic stem cell transplantation, comprising administering to the subject:
(a) an effective amount of an immunoconjugate comprising an antibody, or an antigen binding fragment thereof, linked to at least one pyrrolobenzodiazepine moiety, wherein the antibody, or antigen binding fragment thereof, specifically binds CD300f; and
(b) an effective amount of a lymphodepleting agent.

An alternative eighth aspect provides:
(a) an immunoconjugate comprising an antibody, or an antigen binding fragment thereof, linked to at least one pyrrolobenzodiazepine moiety, wherein the antibody, or antigen binding fragment thereof, specifically binds CD300f; and
(b) a lymphodepleting agent,
   for use in preparing a subject for haematopoietic stem cell transplantation; or use of an immunoconjugate comprising an antibody, or an antigen binding fragment thereof, linked to at least one pyrrolobenzodiazepine moiety, wherein the antibody, or antigen binding fragment thereof, specifically binds CD300f, in the manufacture of a medicament for preparing a subject for haematopoietic stem cell transplantation, wherein the subject is being, has been, or is to be, administered a lymphodepleting agent.

A ninth aspect provides a method of preparing a subject for haematopoietic stem cell transplantation, comprising administering to the subject:
(a) an effective amount of an immunoconjugate comprising an antibody, or an antigen binding fragment thereof, linked to at least one cytotoxic agent, wherein the antibody, or antigen binding fragment thereof, specifically binds CD300f; and
(b) an effective amount of fludarabine or a pharmaceutically acceptable salt thereof.

An alternative ninth aspect provides:
(a) an immunoconjugate comprising an antibody, or an antigen binding fragment thereof, linked to at least one cytotoxic agent, wherein the antibody, or antigen binding fragment thereof, specifically binds CD300f; and
(b) fludarabine or a pharmaceutically acceptable salt thereof, for use in preparing a subject for haematopoietic stem cell transplantation; or use of an immunoconjugate comprising an antibody, or an antigen binding fragment thereof, linked to at least one cytotoxic agent, wherein the antibody, or antigen binding fragment thereof, specifically binds CD300f, in the manufacture of a medicament for preparing a subject for haematopoietic stem cell transplantation, wherein the subject is being, has been, or is to be, administered fludarabine, or a pharmaceutically acceptable salt thereof.

A tenth aspect provides a method of preparing a subject for haematopoietic stem cell transplantation, comprising administering to the subject:
(a) an effective amount of an immunoconjugate comprising an antibody, or an antigen binding fragment thereof, linked to at least one pyrrolobenzodiazepine moiety, wherein the antibody, or antigen binding fragment thereof, specifically binds CD300f;
(b) and an effective amount of fludarabine or a pharmaceutically acceptable salt thereof.

An alternative tenth aspect provides:
(a) an immunoconjugate comprising an antibody, or an antigen binding fragment thereof, linked to at least one pyrrolobenzodiazepine moiety, wherein the antibody, or antigen binding fragment thereof, specifically binds CD300f; and
(b) fludarabine or a pharmaceutically acceptable salt thereof,
   for use in preparing a subject for haematopoietic stem cell transplantation; or use of an immunoconjugate comprising an antibody, or an antigen binding fragment thereof, linked to at least one pyrrolobenzodiazepine moiety, wherein the antibody, or antigen binding fragment thereof, specifically binds CD300f, in the manufacture of a medicament for preparing a subject for haematopoietic stem cell transplantation, wherein the subject is being, has been, or is to be, administered fludarabine, or a pharmaceutically acceptable salt thereof.

An eleventh aspect provides a method of preparing a subject for haematopoietic stem cell transplantation, comprising administering to the subject:
(1) an effective amount of an immunoconjugate comprising an antibody, or antigen binding fragment thereof, linked to at least one cytotoxic agent, wherein the antibody, or antigen binding fragment thereof, specifically binds CD300f and comprises:
   (a) a heavy chain variable region which comprises:
      (i) an amino acid sequence that is at least 70% identical to the amino acid sequence of the amino acid sequence represented by SEQ ID NO: 1; and/or
      (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
   (b) a light chain variable region which comprises:
      (i) an amino acid sequence that is at least 70% identical to the amino acid sequence represented by SEQ ID NO: 5; and/or
      (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8; and
(2) an effective amount of a lymphodepleting agent, typically fludarabine or a pharmaceutically acceptable salt thereof.

An alternative eleventh aspect provides:
(1) an immunoconjugate comprising an antibody, or an antigen binding fragment thereof, linked to at least one cytotoxic agent; and
(2) a lymphodepleting agent, typically fludarabine or a pharmaceutically acceptable salt thereof,
   for use in preparing a subject for haematopoietic stem cell transplantation, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8;
      or use of an immunoconjugate comprising an antibody, or an antigen binding fragment thereof, linked to at least one cytotoxic agent, in the manufacture of a medicament for preparing a subject for haematopoietic stem cell transplantation, wherein the subject is being, has been, or is to be, administered a lymphodepleting agent, typically fludarabine or a pharmaceutically acceptable salt thereof, and wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

A twelfth aspect provides a method of preparing a subject for haematopoietic stem cell transplantation, comprising administering to the subject:
(1) an effective amount of an immunoconjugate comprising an antibody, or antigen binding fragment thereof, linked to at least one pyrrolobenzodiazepine moiety, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8; and
(2) an effective amount of a lymphodepleting agent, typically fludarabine or a pharmaceutically acceptable salt thereof.

An alternative twelfth aspect provides:
(1) an immunoconjugate comprising an antibody, or an antigen binding fragment thereof, linked to at least one pyrrolobenzodiazepine moiety; and
(2) a lymphodepleting agent, typically fludarabine or a pharmaceutically acceptable salt thereof,
   for use in preparing a subject for haematopoietic stem cell transplantation, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8;
      or use of an immunoconjugate comprising an antibody, or an antigen binding fragment thereof, linked to at least one cytotoxic agent, typically at least one pyrrolobenzodiazepine moiety, in the manufacture of a medicament for preparing a subject for haematopoietic stem cell transplantation, wherein the subject is being, has been, or is to be, administered a lymphodepleting agent, typically fludarabine, or a pharmaceutically acceptable salt thereof, and wherein the antibody, or antigen binding fragment thereof, specifically binds CD300f and comprises:
(a) a heavy chain variable region which comprises:
   is (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

A thirteenth aspect provides a method of preparing a subject for haematopoietic stem cell transplantation, comprising administering to the subject:
(1) an effective amount of an immunoconjugate comprising an antibody, or antigen binding fragment thereof, linked to at least one cytotoxic agent, wherein the antibody, or antigen binding fragment thereof, specifically binds CD300f and comprises:
   (a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
   (b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8; and
(2) an effective amount of a lymphodepleting agent, typically fludarabine or a pharmaceutically acceptable salt thereof.

An alternative thirteenth aspect provides:
(a) an immunoconjugate comprising an antibody, or antigen binding fragment thereof, linked to at least one cytotoxic agent; and
(b) a lymphodepleting agent, typically fludarabine or a pharmaceutically acceptable salt thereof,
   for use in preparing a subject for haematopoietic stem cell transplantation, wherein the antibody, or antigen binding fragment thereof, specifically binds CD300f and comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8; or
   use of an immunoconjugate comprising an antibody, or antigen binding fragment thereof, linked to at least one cytotoxic agent, in the manufacture of a medicament for preparing a subject for haematopoietic stem cell transplantation, wherein the subject is being, has been, or is to be, administered a lymphodepleting agent, typically fludarabine, or a pharmaceutically acceptable salt thereof, and wherein the antibody, or antigen binding fragment thereof, specifically binds CD300f and comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

A fourteenth aspect provides a method of preparing a subject for haematopoietic stem cell transplantation, comprising administering to the subject:
(1) an effective amount of an immunoconjugate comprising an antibody, or antigen binding fragment thereof, linked to at least one pyrrolobenzodiazepine moiety, wherein the antibody, or antigen binding fragment thereof, specifically binds CD300f and comprises:
   (a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
   (b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8; and
(2) an effective amount of a lymphodepleting agent, typically fludarabine or a pharmaceutically acceptable salt thereof.

An alternative fourteenth aspect provides:
(1) an immunoconjugate comprising an antibody, or antigen binding fragment thereof, linked to at least one pyrrolobenzodiazepine moiety; and
(2) a lymphodepleting agent, typically fludarabine or a pharmaceutically acceptable salt thereof,
   for use in preparing a subject for haematopoietic stem cell transplantation, wherein the antibody, or antigen binding fragment thereof, specifically binds CD300f and comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8; or
   use of an immunoconjugate comprising an antibody, or antigen binding fragment thereof, linked to at least one pyrrolobenzodiazepine moiety, in the manufacture of a medicament for preparing a subject for haematopoietic stem cell transplantation, wherein the subject is being, has been, or is to be, administered a lymphodepleting agent, typically fludarabine, or a pharmaceutically acceptable salt thereof, and wherein the antibody, or antigen binding fragment thereof, specifically binds CD300f and comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

A fifteenth aspect provides a pharmaceutical composition for preparing a subject for haematopoietic stem cell transplantation, comprising:
(a) an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f; and
(b) a lymphodepleting agent, typically fludarabine or a pharmaceutically acceptable salt thereof.

A sixteenth aspect provides a pharmaceutical composition for preparing a subject for haematopoietic stem cell transplantation, comprising:
(1) an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f and comprises:
   (a) a heavy chain variable region which comprises:
      (i) an amino acid sequence that is at least 70% identical to the amino acid sequence of the amino acid sequence represented by SEQ ID NO: 1; and/or
      (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
   (b) a light chain variable region which comprises:
      (i) an amino acid sequence that is at least 70% identical to the amino acid sequence represented by SEQ ID NO: 5; and/or
      (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8; and
(2) a lymphodepleting agent, typically fludarabine or a pharmaceutically acceptable salt thereof.

A seventeenth aspect provides a pharmaceutical composition for preparing a subject for haematopoietic stem cell transplantation, comprising:
(1) an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8; and
(2) a lymphodepleting agent, typically fludarabine or a pharmaceutically acceptable salt thereof.

An eighteenth aspect provides a kit for preparing a subject for haematopoietic stem cell transplantation, comprising:
(a) an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f; and
(b) a lymphodepleting agent, typically fludarabine or a pharmaceutically acceptable salt thereof.

A nineteenth aspect provides a kit for preparing a subject for haematopoietic stem cell transplantation, comprising:
(1) an immunoconjugate of any one of the first, second or third aspects; and
(2) a lymphodepleting agent, typically fludarabine or a pharmaceutically acceptable salt thereof.

A twentieth aspect provides a method of depleting haematopoietic stem and progenitor cells in a subject, comprising administering to the subject an effective amount of an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f.

An alternative twentieth aspect provides an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f, for use in depleting haematopoietic stem and progenitor cells in a subject; or use of an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f, in the manufacture of a medicament for depleting haematopoietic stem and progenitor cells in a subject.

A twenty first aspect provides a method of depleting haematopoietic stem and progenitor cells in a subject, comprising administering to the subject an effective amount of the immunoconjugate of any one of the first, second or third aspects.

An alternative twenty first aspect provides an immunoconjugate of any one of the first, second or third aspects for use in depleting haematopoietic stem and progenitor cells in a subject; or use of an immunoconjugate of the first, second or third aspect, in the manufacture of a medicament for depleting haematopoietic stem and progenitor cells in a subject.

A twenty second aspect provides a method of preparing a subject for haematopoietic stem cell transplantation, comprising administering to the subject an effective amount of an immunoconjugate of any one of the first, second or third aspects.

An alternative twenty second aspect provides an immunoconjugate of any one of the first, second or third aspects, for use in preparing a subject for haematopoietic stem cell transplantation; or use of an immunoconjugate of any one of the first, second or third aspects in the manufacture of a medicament for preparing a subject for haematopoietic stem cell transplantation.

A twenty third aspect provides a method of preparing a subject for haematopoietic stem cell transplantation, comprising administering to the subject an effective amount of an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f.

An alternative twenty third aspect provides an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f for use in preparing a subject for haematopoietic stem cell transplantation; or use of an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f in the manufacture of a medicament for preparing a subject for haematopoietic stem cell transplantation.

A twenty fourth aspect provides a method of preparing a subject for haematopoietic stem cell transplantation, comprising administering to the subject an effective amount of an immunoconjugate comprising an antibody, or an antigen binding fragment thereof, linked to at least one cytotoxic agent, typically at least one pyrrolobenzodiazepine moiety, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

An alternative twenty third aspect provides an immunoconjugate comprising an antibody, or an antigen binding fragment thereof, linked to at least one cytotoxic agent, typically at least one pyrrolobenzodiazepine moiety, for use in preparing a subject for haematopoietic stem cell transplantation, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8; or

use of an immunoconjugate comprising an antibody, or an antigen binding fragment thereof, linked to at least one cytotoxic agent, typically at least one pyrrolobenzodiazepine moiety, in the manufacture of a medicament for preparing a subject for haematopoietic stem cell transplantation, wherein the antibody, or antigen binding fragment thereof, specifically binds CD300f and comprises:
(a) a heavy chain variable region which comprises:
   (i) an amino acid sequence that is at least 70% identical to the amino acid sequence of the amino acid sequence represented by SEQ ID NO: 1; and/or
   (ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
   (i) an amino acid sequence that is at least 70% identical to the amino acid sequence represented by SEQ ID NO: 5; and/or
   (ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

### BRIEF DESCRIPTION OF THE DRAWINGS

FIG. 1A is a graph of gene expression of CD33 and CD300f compared using linear regression analysis.

FIG. 1B is a graph of MFI ratios of CD33 and CD300f on patient-derived AML cells (n=33) compared with linear regression analysis.

FIG. 1C is a graph showing CD300f gene expression across HSPC subtypes (****P<0.0001 megakaryocyte/erythroid progenitors vs all other subtypes).

FIG. 1D is a graph showing MFI ratios of CD300f on HSPC from healthy bone marrow (BM).

FIG. 2 is a diagram of the gating strategy to identify HSPC groups via flow cytometry.

FIG. 3 is a graph showing MFI ratios of CD300f on the peripheral blood subsets from healthy donors (n=3). Mean with SEM.

FIG. 4A is a graph showing CD300f gene expression by RNA-seq across multiple organ types from a human dataset (****P<0.0001 blood, spleen or lung vs all organs). Grey bars indicate hematopoietic organs. TPM, transcripts per million.

FIG. 4B is a graph showing CD33 gene expression by RNA-seq across multiple organ types (****P<0.0001 blood, spleen, or lung vs all organs). Grey bars indicate hematopoietic organs. TPM, transcripts per million.

FIG. 5 is a graph showing internalization kinetics of DCR-2 on HL-60 cells (% internalized=relative MFI of total−relative MFI of surface staining).

FIG. 6 is a graph showing comparison of cell toxicity of DCR-2 and CMRF-81 directly conjugated to PBD dimer (DCR-2PBD and CMRF-81PBD) in HL-60, U937, THP-1 and Z-138 cells. Cell lines were incubated with immunoconjugates in RPMI (containing Glutamax, Pen/Strep) plus FCS for 72 hours and the toxicity of the immunoconjugates determined. The graph shows that DCR-2-PBD is efficient at killing CD300f+ AML cell lines but not CD300f− Hodgkin Lymphoma cell lines.

FIG. 7 is a graph showing dose-dependent cytotoxicity of haematopoietic progenitor cell colony forming units using DCR-2 PBD.

FIG. 8 is a graph showing dose-dependent cytotoxicity of DCR-2PBD on myeloid, erythroid and multilineage colony forming units. BFU, burst-forming unit; GEMM, granulocyte, erythroid, macrophage, megakaryocyte; GM, granulocyte macrophage.

FIG. 9 is a graph showing Individual AML colony forming unit inhibition with increasing concentration of DCR-2 PBD (performed in duplicate) (*P<0.05 CFU inhibition at 196.2 pM for CRGH1,

**P<0.01 CFU inhibition at 1.6 pM for CRGH9, ***P<0.001 CFU inhibition at 39.2 pM and 196.2 pM for CRGH9).

FIG. 10A shows the steps for assessing cytotoxicity of DCR-2PBD.

FIG. 10B is a graph showing the time dependent cytotoxicity of DCR-2PBD. Cell lines were incubated with immunoconjugate in RPMI containing FCS for a period up to 96 hours after which viability was determined. The graph shows that DCR-2PBD exhibits rapid cytotoxicity.

FIG. 11A is a schematic diagram showing the approach to measuring bystander killing (Bystander Killing Assay).

FIG. 11B is a graph showing the bystander killing effect of DCR-2PBD. Cell lines HL-60 (CD300f+ve cells), Mino (CD300f−ve cells), or mixed populations of HL-60 and Mino were incubated with various concentrations of DCR-2PBD in RPMI containing FCS for 96 hours following which cell viability was determined. In mixed populations, only Mino viability was determined.

FIG. 12 is a graph showing the effect of DCR-2PBD, CMRF-81PBD and PBS control on monocyte-derived dendritic cell survival.

FIG. 13 shows gating for flow cytometric analysis of fresh peripheral blood dendritic cells following treatment with DCR-2PBD and activation with LPS.

FIG. 14 is histograms showing that DCR-2 does not prevent upregulation of activation markers CD80 and CD83 on dendritic cells stimulated with LPS, and therefore does not inhibit DC activation.

FIG. 15A is a schematic diagram showing the approach for testing the effect of DCR-2PBD on T cell proliferation in a mixed lymphocyte reaction.

FIG. 15B is a graph showing that DCR-2PBD does not reduce T cell proliferation in a mixed lymphocyte reaction. Therefore, DCR-2PBD does not prevent antigen presentation to T cells and allows T cells to divide.

FIG. 16A is a schematic of the approach used to test in vivo efficacy of ADCs in a HL-60 xenograft mouse model. Mice were injected with 5×10⁶ HL-60 cells.

FIG. 16B is a graph showing survival of HL-60 xenograft mice following treatment with PBS, CMRF-81PBD or DCR-2PBD.

FIG. 17 is a graph showing effect on U937 tumour volume following treatment of U937 NSG xenograft mice with PBS, CMRF-81PBD or DCR-2PBD at 150 and 300 μg/kg.

FIG. 18 is a graph showing survival of U937 xenograft mice following treatment with PBS, CMRF-81PBD or DCR-2PBD at 150 and 300 μg/kg.

FIG. 19 is a graph showing the dose dependent cytotoxicity of DCR-2PBD alone, fludarabine alone, or a combination of DCR-2PBD and fludarabine, on HL-60 cells.

FIG. 20 are graphs showing (A) Total cell count of CD34+ and CD34+CD38−CD90+ cells in humanized NSG mouse BM 7 days after injection of DCR-2-PBD (n=6) or isotype-PBD (n=5) (**P<0.01 reduction in CD34+ and CD34+CD38−CD90+ cells in the DCR-2-PBD cohort compared with the isotype control cohort); and (B) Cell count per microliter of blood from DCR-2-PBD and isotype-PBD treated humanized mice (**P<0.01 reduction in myeloid cells in peripheral blood of DCR-2-PBD cohort compared with isotype-PBD cohort).

FIG. 21 is a graph showing BM enumeration of primary AML cells 6 days after injection of mice treated with isotype-PBD and DCR-2-PBD (n=6 DCR-2-PBD, n=5 isotype-PBD) (*P<0.05 reduction of primary AML cells in bone marrow, DCR-2-PBD cohort compared with the isotype-PBD cohort).

### DETAILED DESCRIPTION

CD300f is a member of the CD300 family of immunoregulatory molecules encoded by a gene complex on human chromosome 17q25. CD300f is a transmembrane glycoprotein with a cytoplasmic region that contains both inhibitory immunoreceptor tyrosine inhibitory motifs (ITIMs) and phosphatidylinositide-3-kinase (PI3K) phosphorylation sites. Like other members of the CD300 family, CD300f is a transmembrane glycoprotein with a single Ig-like extracellular domain.

The inventors have previously isolated a monoclonal antibody which binds specifically to the extracellular domain of CD300f. The monoclonal antibody is referred to herein as DCR-2 and described in WO2018/094460. A hybridoma producing DCR-2 was deposited at CellBank Australia, 214 Hawkesbury Rd., Westmead, NSW 2145, Australia, under the Budapest Treaty on 27 Sep. 2016 and designated accession number CBA20160029.

DCR-2 binds multiple isoforms of CD300f that are expressed by AML and CD34⁺CD38⁻ leukemic stem cells (LSCs).

The inventors have now found that DCR-2 is effective in killing leukemic cells when linked to one or more pyrrolobenzodiazepine moieties, such as one or more pyrrolobenzodiazepine dimers.

Accordingly, one aspect provides an immunoconjugate comprising an antibody, or antigen binding fragment thereof, linked to at least one pyrrolobenzodiazepine moiety, more typically at least one pyrrolobenzodiazepine dimer, wherein the antibody, or antigen binding fragment thereof, specifically binds to an extracellular domain of CD300f and comprises:

(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and

(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

Typically, the pyrrolobenzodiazepine moiety is a pyrrolobenzodiazepine dimer.

The PBD dimer may be linked to the antibody, or antigen binding fragment thereof, described herein by any methods known in the art. Methods for linking PBD dimers to antibodies are known in the art and described in, for example, WO 2011/130613; WO 2011/130616; WO 2011/130578; WO 2016/064749; and US20160256561.

In one embodiment, the immunoconjugate comprises the following formula I:

Ab-(L-P)m, (I)

or a pharmaceutically acceptable salt or solvate thereof,

wherein:

Ab is an antibody, or antigen binding fragment thereof, as described herein;

L is a linker;

P is a pyrrolobenzodiazepine dimer; and

m is an integer selected from 1 to 10. Typically, m is 1, 2, 3, or 4.

In one embodiment, the immunoconjugate comprises the following formula Ia:

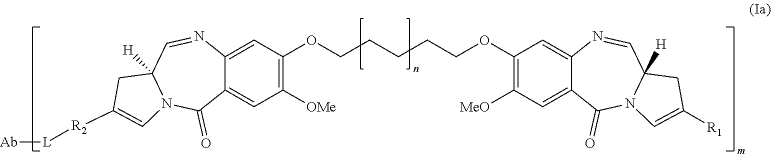

or a pharmaceutically acceptable salt or solvate thereof,

wherein:

Ab is an antibody, or antigen binding fragment thereof, as described herein;

R₂ is C₁-C₆ alkylene or

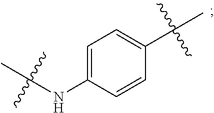 [SMILES: *Nc1ccc(*)cc1]

L is a linker or is absent;

R₁ is C₁-C₆ alkyl (e.g., methyl) or

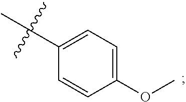 [SMILES: *c1ccc(OC)cc1]

n is 0 or 1; and

and

m is 1, 2, 3 or 4.

In another embodiment, the immunoconjugate comprises the following formula Ib:

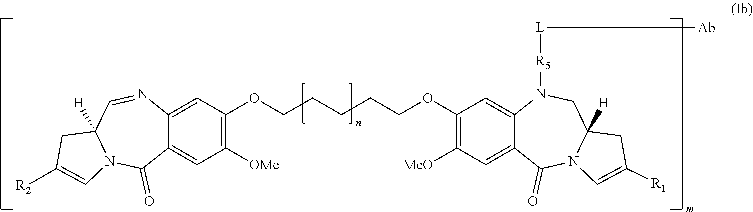

or a pharmaceutically acceptable salt or solvate thereof,

wherein:

Ab is an antibody, or antigen binding fragment thereof, as described herein;

R₂ is C₁-C₆ alkyl (e.g., methyl) or

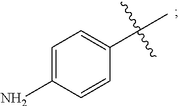 [SMILES: *c1ccc(N)cc1]

L is a linker or is absent;

R₁ is C₁-C₆ alkyl (e.g., methyl) or

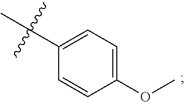 [SMILES: *c1ccc(OC)cc1]

R₅ is

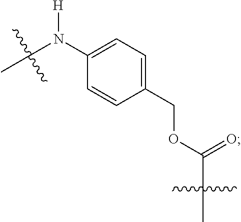 [SMILES: *Nc1ccc(COC(*)=O)cc1]

n is 0 or 1; and

m is 1, 2, 3 or 4.

The linker may be any linker which conjugates the PBD dimer to the antibody without adversely affecting the function of the antibody or the PBD dimer. Linkers suitable for use in preparing antibody drug conjugates are known in the art and described in, for example, US20160303247; US20160367699; US20180147294; U.S. Pat. No. 6,214,345; WO 2016/064749; and US20160256561.

In some embodiments, the linker is a cleavable linker. The cleavable linker may be a linker that is pH or protease sensitive. In some embodiments, the cleavable linker comprises a peptidyl unit that is at least 2 amino acids long. Typically, the cleavable linker is susceptible to an intracellular peptidase or protease enzyme. Examples of peptidyl units include Phe-Leu, Val-Ala or Val-Cit. In some embodiments, the cleavable linker comprises a cleavable peptidyl unit (e.g. Phe-Leu or Val-Ala) that is directly linked to the PBD dimer. In other embodiments, the cleavable peptidyl unit is linked to the PBD dimer via additional units (e.g., spacers).

In some embodiments, a first cleavable peptidyl unit is directly linked to the PBD dimer and a second cleavable peptidyl unit is directly linked to the antibody. In some embodiments, the first and second cleavable peptidyl units are the same. In some embodiments, the first and second cleavable peptide units are different.

The connection between the antibody and the linker may be via a thioether bond, a disulfide bond, an amide bond, or an ester bond. In one embodiment, the linker is connected to the antibody via a thiol group of a cysteine residue of the antibody and a maleimide group of the linker.

In some embodiments, the linker is a non-cleavable linker.

In various embodiments, the linker comprises: C₁-C₆ alkylene; C₁-C₆ heteroalkylene; C₂-C₆ alkenylene; C₂-C₆ heteroalkenylene; C₂-C₆ alkynylene; C₂-C₆ heteroalkynylene; C₃-C₈ cycloalkylene; C₃-C₈ heterocycloalkylene; C₆-C₁₄ arylene; or C₆-C₁₄ heteroarylene.

In one embodiment, the linker in the immunoconjugate has the structure:

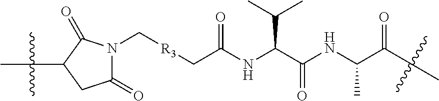

wherein R₃ is selected from the group consisting of:

(a) C₁-C₆ alkylene;

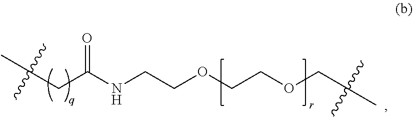

wherein q is an integer from 1 to 6, and r is an integer from 1 to 15;

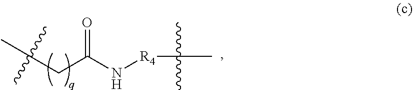

wherein q is an integer from 1 to 6, and R₄ is a C₁-C₆ alkylene; and

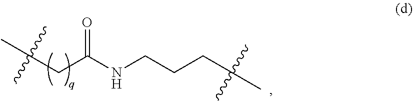

wherein q is an integer from 1 to 6.

In one embodiment, the linker in the immunoconjugate has the structure:

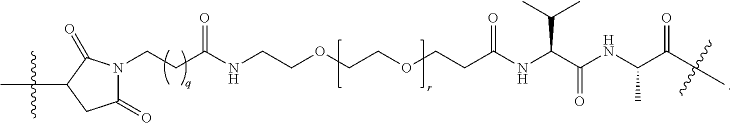

wherein q is an integer from 1 to 6; and r is an integer from 1 to 15, typically r is an integer from 1 to 12, more typically r is an integer selected from the group consisting of 1, 2, 3, 4, 5, 6, 7, 8, 9 and 10. In one embodiment, r is 2.

In one embodiment, the linker in the immunoconjugate has the structure:

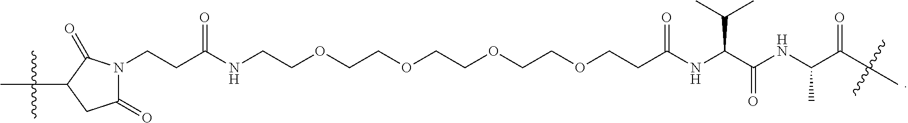 [SMILES: *C(=O)[C@H](C)NC(=O)[C@@H](NC(=O)CCOCCOCCOCCOCCNC(=O)CCN1C(=O)CC(*)C1=O)C(C)C]

In one embodiment, the immunoconjugate has the following structure:

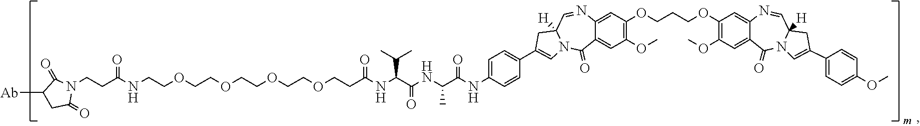

or a pharmaceutically acceptable salt or solvate thereof,
wherein:
Ab is an antibody, or antigen binding fragment thereof, as described herein; and
m is 1, 2, 3 or 4.

As shown in the Examples, the inventors have found that although DCR-2 alone does not prolong survival or reduce disease burden in a NOS/SCID mouse model of AML, when coupled with a pyrrolobenzodiazepine dimer, the resulting immunoconjugate:
(a) is efficient at killing AML cells;
(b) causes bystander killing of CD300f −ve cells;
(c) prolongs survival in HL-60 inoculated NSG mice greater than CMRF-81 mAb coupled with PBD dimer;
(d) slows tumour growth and prolongs survival in U937 inoculated mice greater than CMRF-81 mAb coupled with PBD dimer;
(e) synergizes with fludarabine to provide an enhanced cytotoxic effect.

The present disclosure relates in one aspect to an immunoconjugate comprising an antibody, or antigen binding fragment thereof, coupled to at least one pyrrolobenzodiazepine moiety, typically one or more pyrrolobenzodiazepine dimers, wherein the antibody, or antigen binding fragment thereof, specifically binds to an extracellular domain of CD300f, wherein the antibody or antigen binding fragment thereof comprises:

(a) a heavy chain variable region which comprises:

(i) a complementarity determining region 1 (CDR1) that is identical to CDR1 of the heavy chain variable region of monoclonal antibody DCR-2, a complementarity determining region 2 (CDR2) that is identical to CDR2 of the heavy chain variable region of monoclonal antibody DCR-2, and/or a complementarity determining region 3 (CDR3) that is identical to CDR3 of the heavy chain variable region of monoclonal antibody DCR-2; and

(b) a light chain variable region which comprises:

(i) a complementarity determining region 1 (CDR1) that is identical to CDR1 of the light chain variable region of monoclonal antibody DCR-2, a complementarity determining region 2 (CDR2) that is identical to CDR2 of the light chain variable region of monoclonal antibody DCR-2, and/or a complementarity determining region 3 (CDR3) that is identical to CDR3 of the light chain variable region of monoclonal antibody DCR-2.

The amino acid and nucleic acid sequences referred to in the sequence listing are set out in Table 1.

TABLE 1
Antibody sequences referred to in the sequence listing

| SEQ ID NO: | Description |
|---|---|
| 1 | amino acid sequence of heavy chain variable region of DCR-2 |
| 2 | amino acid sequence of CDR1 of heavy chain variable region |
| | of DCR-2 |
| 3 | amino acid sequence of CDR2 of heavy chain variable region |
| | of DCR-2 |
| 4 | amino acid sequence of CDR3 of heavy chain variable region |
| | of DCR-2 |
| 5 | amino acid sequence of light chain variable region of DCR-2 |
| 6 | amino acid sequence of CDR1 of light chain variable region of |
| | DCR-2 |
| 7 | amino acid sequence of CDR2 of light chain variable region of |
| | DCR-2 |
| 8 | amino acid sequence of CDR3 of light chain variable region of |
| | DCR-2 |
| 9 | nucleotide sequence of heavy chain variable region of DCR-2 |
| 10 | nucleotide sequence of light chain variable region of DCR-2 |
| 11 | codon optimized nucleotide sequence encoding chimeric |
| | antibody heavy chain comprising the heavy chain variable |
| | region of DCR-2 combined with the constant region of the |
| | heavy chain of human anti-TNP IgG1. |
| 12 | codon optimized nucleotide sequence encoding chimeric |
| | antibody light chain comprising the light chain variable region |
| | of DCR-2 combined with the constant region of the kappa |
| | chain of human anti-TNP IgG1. |
| 13 | amino acid sequence encoded by SEQ ID NO: 11. |
| 14 | amino acid sequence encoded by SEQ ID NO: 12. |
| 15 | amino acid sequence of humanized heavy chain variable |
| | region of DCR-2 |

The amino acid sequence of the heavy chain variable region (V_{H}) of DCR-2 is represented by the amino acid sequence:

(SEQ ID NO: 1)
M E S G G G L V Q P G G P L K L S C A A S G F
G F S G S W M S W V R Q A P G K G L E W I G Q
I N P D S S T I N Y T P S L K D K F I I S R D
N A K N T L Y L Q I N K V R S E D T A L Y Y C
A R R G F F E G Y S A W F A Y W.

The amino acid sequence of CDR1 of the heavy chain variable region of DCR-2 is represented by the amino acid sequence GFGFSGSW (SEQ ID NO: 2).

The amino acid sequence of CDR2 of the heavy chain variable region of DCR-2 is represented by the amino acid sequence INPDSSTI (SEQ ID NO: 3).

The amino acid sequence of CDR3 of the heavy chain variable region of DCR-2 is represented by the amino acid sequence ARRGFFEGYSAWFAY (SEQ ID NO: 4).

The amino acid sequence of the light chain variable region (V_{L}) of DCR-2 is represented by the amino acid sequence:

(SEQ ID NO: 5)
I L M T Q T P K F L L V S A G D R V T I T C K
A S Q S V S N D V A W Y Q Q K P G Q S P S L L
I Y Y A S N R N T G V P D R F T G S G Y E T D
F T F T I S T V Q A E D L A V Y F C Q Q D Y T
S P W T F G G G.

The amino acid sequence of CDR1 of the light chain variable region of DCR-2 is represented by the amino acid sequence QSVSND (SEQ ID NO: 6).

The amino acid sequence of CDR2 of the light chain variable region of DCR-2 is represented by the amino acid sequence YAS (SEQ ID NO: 7).

The amino acid sequence of CDR3 of the light chain variable region of DCR-2 is represented by the amino acid sequence QQDYTSPWT (SEQ ID NO: 8).

The present disclosure therefore relates in one aspect to an immunoconjugate comprising an antibody, or antigen binding fragment thereof, linked to at least one pyrrolobenzodiazepine moiety, typically one or more pyrrolobenzodiazepine dimers, wherein the antibody or antigen binding fragment thereof specifically binds to an extracellular domain of CD300f and comprises:

(a) a heavy chain variable region which comprises:

(i) an amino acid sequence that is at least 70%, typically at least 75%, 80%, 85%, 90%, 95%, 96%, 97%, 98%, or 99% identical to the amino acid sequence represented by SEQ ID NO: 1; or

(ii) a complementarity determining region 1 (CDR1) that comprises the amino acid sequence GFGFSGSW (SEQ ID NO: 2), a complementarity determining region 2 (CDR2) that comprises the amino acid sequence INPDSSTI (SEQ ID NO: 3), and/or a complementarity determining region 3 (CDR3) that comprises the amino acid sequence ARRGFFEGYSAWFAY (SEQ ID NO: 4); and/or

(b) a light chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 5; or

(ii) a complementarity determining region 1 (CDR1) that comprises the amino acid sequence QSVSND (SEQ ID NO: 6), a complementarity determining region 2 (CDR2) that comprises the amino acid sequence YAS (SEQ ID NO: 7), and/or a complementarity determining region 3 (CDR3) that comprises the amino acid sequence QQDYTSPWT (SEQ ID NO: 8).

In one embodiment, the antibody or antigen binding fragment thereof comprises:

(a) a heavy chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 1; or

(ii) a complementarity determining region 1 (CDR1) that comprises the amino acid sequence represented by SEQ ID NO: 2, a complementarity determining region 2 (CDR2) that comprises the amino acid sequence represented by SEQ ID NO: 3, and/or a complementarity determining region 3 (CDR3) that comprises the amino acid sequence represented by SEQ ID NO: 4; and

(b) a light chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 5; or

(ii) a complementarity determining region 1 (CDR1) that comprises the amino acid sequence represented by SEQ ID NO: 6, a complementarity determining region 2 (CDR2) that comprises the amino acid sequence represented by SEQ ID NO: 7, and/or a complementarity determining region 3 (CDR3) that comprises the amino acid sequence represented by SEQ ID NO: 8.

In one embodiment, the antibody or antigen binding fragment thereof comprises:

(a) a heavy chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 1; and/or

(b) a light chain variable region which comprises:

(i) an amino acid sequence that is at least 70% identical, typically at least 75%, 80%, 85%, 90%, 95%, 96%, 97%, 98%, or 99% identical, to the amino acid sequence represented by SEQ ID NO: 5.

In one embodiment, the antibody, or antigen binding fragment thereof, comprises:

(a) a heavy chain variable region which comprises:

(i) a complementarity determining region 1 (CDR1) that comprises the amino acid sequence represented by SEQ ID NO: 2, a complementarity determining region 2 (CDR2) that comprises the amino acid sequence represented by SEQ ID NO: 3, and a complementarity determining region 3 (CDR3) that comprises the amino acid sequence represented by SEQ ID NO: 4; or

(b) a light chain variable region which comprises:

(i) a complementarity determining region 1 (CDR1) that comprises the amino acid sequence represented by SEQ ID NO: 6, a complementarity determining region 2 (CDR2) that comprises the amino acid sequence represented by SEQ ID NO: 7, and a complementarity determining region 3 (CDR3) that comprises the amino acid sequence represented by SEQ ID NO: 8.

In one embodiment, the antibody, or antigen binding fragment thereof, comprises:

(a) a heavy chain variable region which comprises:

(i) a complementarity determining region 1 (CDR1) that comprises the amino acid sequence represented by SEQ ID NO: 2, a complementarity determining region 2 (CDR2) that comprises the amino acid sequence represented by SEQ ID NO: 3, and a complementarity determining region 3 (CDR3) that comprises the amino acid sequence represented by SEQ ID NO: 4; and

(b) a light chain variable region which comprises:

(i) a complementarity determining region 1 (CDR1) that comprises the amino acid sequence represented by SEQ ID NO: 6, a complementarity determining region 2 (CDR2) that is identical to the amino acid sequence represented by SEQ ID NO: 7, and a complementarity determining region 3 (CDR3) that comprises the amino acid sequence represented by SEQ ID NO: 8.

In one embodiment, the antibody, or antigen binding fragment thereof, comprises a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1. In one embodiment, the heavy chain variable region comprises an amino acid sequence that is 100% identical to the amino acid sequence represented by SEQ ID NO: 1.

In one embodiment, the antibody, or antigen binding fragment thereof, comprises a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5. In one embodiment, the light chain variable region comprises an amino acid sequence that is 100% identical to the amino acid sequence represented by SEQ ID NO: 5.

In various embodiments, the antibody, or antigen binding fragment thereof, which specifically binds CD300f comprises:
(a) a heavy chain variable region comprising the amino acid sequence represented by SEQ ID NO: 1;
(b) a heavy chain variable region comprising an amino acid sequence that is at least 70% identical, typically at least 75%, 80%, 85%, 90%, 95%, 96%, 97%, 98%, or 99% identical, to the amino acid sequence represented by SEQ ID NO: 1;
(c) a light chain variable region comprising the amino acid sequence represented by SEQ ID NO: 5;
(d) a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5;
(e) a heavy chain variable region comprising the amino acid sequence represented by SEQ ID NO: 1, and a light chain variable region comprising the amino acid sequence represented by SEQ ID NO: 5;
(f) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5;
(g) a heavy chain CDR1 comprising the amino acid sequence represented by SEQ ID NO: 2;
(h) a heavy chain CDR2 comprising the amino acid sequence represented by SEQ ID NO: 3;
(i) a heavy chain CDR3 comprising the amino acid sequence represented by SEQ ID NO: 4;
(j) a heavy chain CDR1 comprising the amino acid sequence represented by SEQ ID NO: 2 and a heavy chain CDR2 comprising the amino acid sequence represented by SEQ ID NO: 3;
(k) a heavy chain CDR1 comprising the amino acid sequence represented by SEQ ID NO: 2 and a heavy chain CDR3 comprising the amino acid sequence represented by SEQ ID NO: 4;
(l) a heavy chain CDR2 comprising the amino acid sequence represented by SEQ ID NO: 3 and a heavy chain CDR3 comprising the amino acid sequence represented by SEQ ID NO: 4;
(m) a heavy chain CDR1 comprising the amino acid sequence represented by SEQ ID NO: 2, a heavy chain CDR2 comprising the amino acid sequence represented by SEQ ID NO: 3, and a heavy chain CDR3 comprising the amino acid sequence represented by SEQ ID NO: 4;
(n) a light chain CDR1 comprising the amino acid sequence represented by SEQ ID NO: 6;
(o) a light chain CDR2 comprising the amino acid sequence represented by SEQ ID NO: 7;
(p) a light chain CDR3 comprising the amino acid sequence represented by SEQ ID NO: 8;
(q) a light chain CDR1 comprising the amino acid sequence represented by SEQ ID NO: 6, and a light chain CDR2 comprising the amino acid sequence represented by SEQ ID NO: 7;
(r) a light chain CDR1 comprising the amino acid sequence represented by SEQ ID NO: 6, and a light chain CDR3 comprising the amino acid sequence represented by SEQ ID NO: 8;
(s) a light chain CDR2 comprising the amino acid sequence represented by SEQ ID NO: 7, and a light chain CDR3 comprising the amino acid sequence represented by SEQ ID NO: 8;
(t) a light chain CDR1 comprising the amino acid sequence represented by SEQ ID NO: 6, a light chain CDR2 comprising the amino acid sequence represented by SEQ ID NO: 7, and a light chain CDR3 comprising the amino acid sequence represented by SEQ ID NO: 8;
(u) a heavy chain CDR1 comprising the amino acid sequence represented by SEQ ID NO: 2, and a light chain CDR1 comprising the amino acid sequence represented by SEQ ID NO: 6;
(v) a heavy chain CDR2 comprising the amino acid sequence represented by SEQ ID NO: 3, and a light chain CDR2 comprising the amino acid sequence represented by SEQ ID NO: 7;
(w) a heavy chain CDR3 comprising the amino acid sequence represented by SEQ ID NO: 4, and a light chain CDR3 comprising the amino acid sequence represented by SEQ ID NO: 8;
(x) a heavy chain CDR1 comprising the amino acid sequence represented by SEQ ID NO: 2, a heavy chain CDR2 comprising the amino acid sequence represented by SEQ ID NO: 3, a light chain CDR1 comprising the amino acid sequence represented by SEQ ID NO: 6, and a light chain CDR2 comprising the amino acid sequence represented by SEQ ID NO: 7;
(y) a heavy chain CDR1 comprising the amino acid sequence represented by SEQ ID NO: 2, a heavy chain CDR3 comprising the amino acid sequence represented by SEQ ID NO: 4, a light chain CDR1 comprising the amino acid sequence represented by SEQ ID NO: 6, and a light chain CDR3 comprising the amino acid sequence represented by SEQ ID NO: 8;
(z) a heavy chain CDR2 comprising the amino acid sequence represented by SEQ ID NO: 3, a heavy chain CDR3 comprising the amino acid sequence represented by SEQ ID NO: 4, a light chain CDR2 comprising the amino acid sequence represented by SEQ ID NO: 7, and a light chain CDR3 comprising the amino acid sequence represented by SEQ ID NO: 8;
(aa) a heavy chain CDR1 comprising the amino acid sequence represented by SEQ ID NO: 2, a heavy chain CDR2 comprising the amino acid sequence represented by SEQ ID NO: 3, a heavy chain CDR3 comprising the amino acid sequence represented by SEQ ID NO: 4, a light chain CDR1 comprising the amino acid sequence represented by SEQ ID NO: 6, a light chain CDR2 comprising the amino acid sequence represented by SEQ ID NO: 7, and a light chain CDR3 comprising the amino acid sequence represented by SEQ ID NO: 8;
(bb) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4;
(cc) a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8;
(dd) a heavy chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4, and a light chain variable region which comprises an amino acid sequence that is at least 70% identical, typically at least 75%, 80%, 85%, 90%, 95%, 96%, 97%, 98%, or 99% identical, to the amino acid sequence represented by SEQ ID NO: 5, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8;
(ee) a heavy chain variable region which comprises an amino acid sequence that is represented by SEQ ID NO: 15, and a light chain variable region which comprises an amino acid sequence that is at least 70% identical, typically at least 75%, 80%, 85%, 90%, 95%, 96%, 97%, 98%, 99% identical, to the amino acid sequence represented by SEQ ID NO: 5.

In one embodiment, the antibody, or antigen binding fragment thereof, that specifically binds to CD300f, comprises a heavy chain comprising the amino acid sequence represented by SEQ ID NO: 13.

In one embodiment, the antibody, or antigen binding fragment thereof, that specifically binds to CD300f, comprises a light chain comprising the amino acid sequence represented by SEQ ID NO: 14.

In one embodiment, the antibody, or antigen binding fragment thereof, that specifically binds to CD300f, comprises a heavy chain comprising the amino acid sequence represented by SEQ ID NO: 13 and a light chain comprising the amino acid sequence represented by SEQ ID NO: 14.

In one embodiment, the antibody, or antigen binding fragment thereof, that specifically binds to CD300f, comprises a heavy chain variable region which comprises an amino acid sequence that is at least 90% identical to the amino acid sequence represented by SEQ ID NO: 1, and a light chain variable region which comprises an amino acid sequence that is at least 90% identical to the amino acid sequence represented by SEQ ID NO: 5.

In one embodiment, the antibody, or antigen binding fragment thereof, that specifically binds to CD300f, comprises a heavy chain variable region which comprises an amino acid sequence that is at least 95% identical to the amino acid sequence represented by SEQ ID NO: 1, and a light chain variable region which comprises an amino acid sequence that is at least 95% identical to the amino acid sequence represented by SEQ ID NO: 5.

In one embodiment, the antibody, or antigen binding fragment thereof, that specifically binds to CD300f, comprises: a heavy chain variable region which comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

In one embodiment, the antibody, or antigen binding fragment thereof, that specifically binds to CD300f, comprises: a heavy chain variable region which comprises an amino acid sequence that is at least 90% identical to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises an amino acid sequence that is at least 90% identical to the amino acid sequence represented by SEQ ID NO: 1, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

In one embodiment, the antibody, or antigen binding fragment thereof, that specifically binds to CD300f, comprises: a heavy chain variable region which comprises an amino acid sequence that is at least 95% identical to the amino acid sequence represented by SEQ ID NO: 1, and comprises a CDR1 comprising the amino acid sequence represented by SEQ ID NO: 2, a CDR2 comprising the amino acid sequence represented by SEQ ID NO: 3, and a CDR3 comprising the amino acid sequence represented by SEQ ID NO: 4; and a light chain variable region which comprises an amino acid sequence that is at least 95% identical to the amino acid sequence represented by SEQ ID NO: 1, and comprises a CDR1 comprising the amino acid sequence represented by SEQ ID NO: 6, a CDR2 comprising the amino acid sequence represented by SEQ ID NO: 7, and a CDR3 comprising the amino acid sequence represented by SEQ ID NO: 8.

The percent identity between two amino acid sequences can be determined using any alignment algorithms known in the art, including for example, the FASTA package of sequence analysis programs (Lipman & Pearson, (1985) Science 227(4693): 1435-1441); BLAST (Altschul et al. J. Mol. Biol. 215(3):403-410.

DCR-2 may be obtained from a hybridoma deposited under the Budapest Treaty on 27 Sep. 2016 at CellBank Australia, 214 Hawkesbury Road, Westmead, NSW 2145, Australia, and designated accession no. CBA20160029. The hybridoma deposited under the Budapest Treaty and designated accession no. CNA20160029 expresses DCR-2.

An immunoconjugate refers to an antigen binding protein linked to a moiety, such as a drug. Typically, the immunoconjugate is an antibody drug conjugate (ADC).

An antibody refers to an immunoglobulin molecule capable of specifically binding to an antigen. The antibody may be recombinant or modified, including chimeric, humanized, deimmunised, CDR-grafted, bi-specific, human. A full-length antibody typically comprises two light chains covalently linked to two heavy chains. Each heavy chain of the full-length antibody comprises a heavy chain variable region and a heavy chain constant region. Each light chain of a full-length antibody comprises a light chain variable region and a light chain constant region. Full length antibodies may be any of the following type: IgG, IgM, IgE, IgD, IgA. In one embodiment, the antibody is IgG.

As used herein, an “antigen binding fragment” of an antibody comprises an antigen binding domain of the antibody, and typically comprises a portion of the antibody that specifically binds the same epitope as the full-length antibody. Typically, the antibody fragment of an antibody comprises portions of the variable region of the heavy and/or the light chain of the antibody. Typically, the antigen binding fragment comprises the CDR1, 2 and/or 3 region of the heavy chain variable region and/or the CDR1, 2 and/or 3 region of the light chain variable region. More typically, the antigen binding fragment comprises the CDR1, 2 and 3 region of the heavy chain variable region and/or the CDR1, 2 and 3 region of the light chain variable region. Still more typically, the antigen binding fragment comprises the CDR1, 2 and 3 region of the heavy chain variable region, and the CDR1, 2 and 3 region of the light chain variable region. In some embodiments, the antigen binding fragment of an antibody comprises the heavy chain variable region and the light chain variable region of an antibody. The portions of the heavy and light chain variable regions may be on separate polypeptide chains, such as Fv fragments, or in a single polypeptide chain in which the light chain and heavy chain variable regions are connected by a peptide linker (“scFv proteins”). Examples of antigen binding fragments of an antibody may include F(ab′)₂, Fab′, Fab, Fv, sFv, scFv, and the like.

As used herein, an antigen binding fragment of an antibody encompasses one or more polypeptides which comprise an antigen binding domain of the antibody, such as an F(ab′)₂, Fab′, Fab, Fv, sFv, or scFv.

An “antigen binding domain” refers to a region of an antibody that is capable of specifically binding to an antigen. Typically, the antigen binding domain comprises CDR1, CDR2 and/or CDR3 from the light chain variable region, and/or CDR1, CDR2 and/or CDR3 from the heavy chain variable region, of an antibody. More typically, the antigen binding domain comprises CDR1, CDR2 and CDR3 from the light chain variable region, and/or CDR1, CDR2 and/or CDR3 from the heavy chain variable region, of an antibody. Still more typically, the antigen binding domain comprises CDR1, CDR2 and CDR3 from the light chain variable region, and CDR1, CDR2 and CDR3 from the heavy chain variable region, of an antibody.

The term “variable region” refers to the portion of the light and/or heavy chain of an antibody that is capable of specifically binding to an antigen. The variable region comprises the complementarity determining regions (CDRs) and the framework regions (FRs). Framework regions are those variable regions other than the complementarity determining regions.

The term “complementarity determining region” refers to one of three amino acid sequences of the variable region of the light chain variable region and/or heavy chain variable region of an antibody that is largely responsible for the ability of the antibody to bind specifically to an antigen. The three complementarity determining regions of the variable region of the light and heavy chain are referred to as CDR1, CDR2 and CDR3.

Methods for determining the CDR regions and the framework (FR) regions of the variable region of the light and heavy chain are known in the art. For example, the amino acid positions assigned to CDRs and FRs may be defined according to Kabat Sequences of Proteins of Immunological Interest, National Institute of Health, Bethesda, Md., 1987 and 1991; Enhanced Clothia Numbering Scheme; Clothia and Lesk J. Mol. Biol. 196:901-917; Clothia et al. Nature 342: 877-883; Honnegher and Plukthun, J. Mol. Biol. 309: 657-670. The antibody, or antigen binding fragment thereof, specifically binds to the extracellular domain of CD300f. As used herein, “an antibody, or antigen binding fragment thereof, that specifically binds to an extracellular domain of CD300f” is an antibody or antigen binding fragment thereof that associates with the extracellular domain of CD300f more frequently, more rapidly, for greater length of time, or with greater affinity, that with other antigens.

The variable domains from antibodies may be cloned using conventional techniques that are known in the art and described in, for example, Sambrook and Russell, Eds, Molecular Cloning: A Laboratory Manual, 3^{rd} Ed, vols. 1-3, Cold Spring Harbor Laboratory Press, 2001. In general, the light chain variable region and heavy chain variable region sequences for antibodies, such as murine antibodies, can be obtained by a variety of molecular cloning procedures, such as RT-PCR, 5′-RACE, and cDNA library screening.

As used herein, a chimeric antibody is an antibody protein that comprises the complementarity determining regions (CDRs), typically the variable regions, of an antibody derived from one species, typically a mouse antibody, while the constant domains of the antibody molecule, and in some embodiments, the framework regions (FR), are derived from another species, such as a human.

A humanised antibody is a form of chimeric antibody in which the amino acid sequence of the CDRs is from an antibody from one species; e.g., a mouse antibody, and the amino acid sequence of the constant regions, and typically the framework regions, is from a human antibody.

In one embodiment, the antibody or antigen binding fragment thereof is a chimeric antibody. The chimeric antibody comprises the complementarity-determining regions (CDRs), and typically framework regions (FR), of DCR-2. The chimeric antibody may comprise the light and heavy chain constant regions of a human antibody. The use of antibody components derived from chimerized monoclonal antibodies reduces potential problems associated with the immunogenicity of murine constant regions. Typically, the antibody is a humanised antibody. Humanization of murine antibodies and antibody fragments is known to those skilled in the art, and described in, for example, U.S. Pat. Nos. 5,225,539; 6,054,297; and 7,566,771. For example, humanized monoclonal antibodies may be produced by transferring murine complementary determining regions from heavy and light variable chains of the mouse immunoglobulin into a human variable domain, and then, substituting human residues in the framework regions of the murine counterparts. The use of human framework region sequences, in addition to human constant region sequences, further reduces the chance of inducing HAMA reactions.

Antibodies can be isolated and purified from serum and hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in Methods in Molecular Biology, Vol. 10, pages 79-104 (The Humana Press, Inc. 1992).

In some embodiments, an antigen binding fragment of an antibody includes portions of the variable region of the heavy and/or light chain of the antibody. The portions of the heavy chain variable region and/or light chain variable region may be on separate polypeptide chains, such as Fv fragments, or in a single polypeptide chain in which light and heavy variable regions are connected by a peptide linker (e.g. scFv proteins). Examples of antibody fragments include F(ab′)₂, Fab′, Fab, Fv, sFv, scFv, and the like. Typically, the antibody fragment comprises the CDR1, 2 and 3 region of the heavy chain variable region and/or the CDR1, 2 and 3 region of the light chain variable region. Antibody fragments which recognize specific epitopes can be generated by known techniques. F(ab′)₂ fragments, for example, can be produced by pepsin digestion of the antibody molecule. These and other methods are described, for example, by Coligan at pages 2.8.1-2.8.10 and 2.10.-2.10.4. Alternatively, Fab′ expression libraries can be constructed to allow rapid and easy identification of Fab′ fragments with the desired specificity.

In some embodiments, the antibody, or antigen binding fragment thereof, has a reduced half-life compared to naturally occurring IgG. In haematopoietic stem cell transplantation, it is advantageous to reduce the half-life of the antibody to reduce damage to donor tissue being transplanted. The half-life of an antibody can be reduced, for example, by removing the neonatal Fc receptor (FcRn) recognition site. The FcRn recognition site of an IgG antibody is within the CH2-CH3 region in the Fc portion of IgG. Removal of the CH2-CH3 region of IgG will remove the FcRn recognition site and thereby reduce the half-life of the antibody. Antibody fragments such as F(ab′)₂, Fab′, Fab, Fv, sFv, scFv lack the FcRn recognition site.

Methods for making antibody drug conjugates (ADC) are known in the art, and are described in, for example, Alley et al., Current Opinion in Chemical Biology, 2010 14:1-9; US20160303247; US20160367699; US20180147294; WO 2016/064749; U.S. Pat. No. 9,399,641; U.S. Ser. No. 10/010,624. PBD dimer and linker combinations for conjugating PBD dimers to antibodies, or antigen binding fragments thereof, are described in, for example, WO 2011/130613; WP 2011/130616; WO 2011/130578; and WO 2016/064749. Additionally, kits for preparing ADCs are commercially available from, for example, Levena Biopharma, Calif.

The immunoconjugate described herein may be formulated as a pharmaceutical composition. Accordingly, a further aspect provides a pharmaceutical composition comprising an isolated DCR-2 antibody, or antigen binding fragment thereof, as described herein linked to at least one pyrrolobenzodiazepine molecule, typically one or more pyrrolobenzodiazepine dimers, and a pharmaceutically acceptable carrier.

A “pharmaceutically acceptable carrier” means that it is compatible with the other ingredients of the composition and is not deleterious to a subject. The composition may contain other therapeutic agents as described below, and may be formulated, for example, by employing conventional liquid vehicles or diluents, as well as pharmaceutical additives of a type appropriate to the mode of desired administration (for example, excipients, binders, preservatives, stabilizers, etc.) according to techniques such as those well known in the art of pharmaceutical formulation (See, for example, Remington: The Science and Practice of Pharmacy, 21st Ed., 2005, Lippincott Williams & Wilkins).

The pharmaceutical compositions are typically in the form of a sterile injectable aqueous suspension. This suspension may be formulated according to the known art and contain the active materials in admixture with excipients suitable for the manufacture of aqueous suspensions. Such excipients may include suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectable formulations.

As described in WO2018/094460, DCR-2 binds to cells expressing CD300f, including haematopoietic stem and progenitor cells, AML and leukaemic stem cells.

Accordingly, the immunoconjugate described herein may be used to treat a subject suffering from a condition associated with CD300f expression. Accordingly, a further aspect provides a method of treating a condition associated with CD300f expression, such as AML, comprising administering to a subject in need thereof an effective amount of an immunoconjugate comprising an antibody, or antigen binding fragment thereof, coupled to at least one pyrrolobenzodiazepine molecule, typically one or more pyrrolobenzodiazepine dimers, wherein the antibody or antigen binding fragment thereof specifically binds CD300f and comprises:

(a) a heavy chain variable region which comprises:
(i) an amino acid sequence that is at least 70% identical, typically at least 75%, 80%, 85%, 90%, 95%, 96%, 97%, 98%, or 99% identical to the amino acid sequence represented by SEQ ID NO: 1; and/or
(ii) a complementarity determining region 1 (CDR1) comprising the amino acid sequence represented by SEQ ID NO: 2, a complementarity determining region 2 (CDR2) comprising the amino acid sequence represented by SEQ ID NO: 3, and/or a complementarity determining region 3 (CDR3) comprising the amino acid sequence represented by SEQ ID NO: 4; and/or
   (b) a light chain variable region which comprises:
(i) an amino acid sequence that is at least 70% identical, 70%, typically at least 75%, 80%, 85%, 90%, 95%, 96%, 97%, 98%, or 99% identical to the amino is acid sequence represented by SEQ ID NO: 5; and/or
(ii) a complementarity determining region 1 (CDR1) comprising the amino acid sequence represented by SEQ ID NO: 6, a complementarity determining region 2 (CDR2) comprising the amino acid sequence represented by SEQ ID NO: 7, and/or a complementarity determining region 3 (CDR3) comprising the amino acid sequence represented by SEQ ID NO: 8.

In one embodiment, the condition associated with CD300f expression is a myeloid leukaemia. In one embodiment, the myeloid leukaemia is AML.

In various embodiments, there is provided a method of treating a condition associated with CD300f expression, such as AML, comprising administering to a subject in need thereof an effective amount of the immunoconjugate described herein.

Typically, the immunoconjugate is administered in a pharmaceutically acceptance composition as described herein.

A large proportion of the morbidity associated with allogeneic haematopoietic stem cell transplantation is due to nonspecific toxic conditioning agents which are required to remove recipient Hematopoietic Stem and Progenitor Cells (HSPC) allowing for successful engraftment. The CD300f immunoconjugates described herein effectively depletes HSPC and AML cell lines and colony forming units. As described in the Examples, the CD300f immunoconjugate described herein prolongs the survival of mice xenografts with multiple cell lines and depletes primary AML engrafted after a single injection. In a humanised mouse model a single injection of the antibody conjugate depletes CD34+ HSPC and CD34+ CD38− CD90+ HSC.

The ability to effectively deplete HSPC and AML cells by targeting CD300f means that the CD300f immunoconjugate described herein can be used in conditioning regimens for allogeneic haematopoietic stem cell transplantation, thus avoiding the need for nonspecific toxic conditioning agents such as radiation or alkylating agent therapy.

Accordingly, in one aspect, there is provided a method of preparing a subject for haematopoietic stem cell transplantation, comprising administering to the subject an effective amount of an immunoconjugate comprising an antibody, or antigen binding fragment thereof, which specifically binds CD300f, linked to at least one cytotoxic agent, such as at least one pyrrolobenzodiazepine dimer. In one embodiment, the antibody, or antigen binding fragment thereof, comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

A further aspect provides a method of depleting haematopoietic stem cells in a subject, comprising administering to the subject an effective amount of an immunoconjugate comprising an antibody, or antigen binding fragment thereof, which specifically binds CD300f, linked to at least one cytotoxic agent, such as at least one pyrrolobenzodiazepine dimer. In one embodiment, the antibody, or antigen binding fragment thereof, comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.
As used herein, a cytotoxic agent is a compound that has a cytotoxic effect on a cell when contacted with the cell or introduced into the cell. A cytotoxic effect may be cell death or a reduction in cell viability.

As described in the Examples, the inventors have further found that when the AML cell line HL-60 is treated with DCR-2 linked to a pyrrolobenzodiazepine dimer (DCR-2PBD) and fludarabine, the cytotoxic effect of the combined treatment is greater than the sum of the cytotoxic effect of DCR-2PBD and the cytotoxic effect of fludarabine. In this regard, the inventors have found that the cytotoxic effect of the combination of DCR-2PBD and fludarabine is synergistic.

As the combined effect of DCR-2PBD and fludarabine is synergistic, the inventors envisage that when fludarabine is used in combination with DCR-2PBD, the fludarabine can be used at lower, more tolerated concentrations. The inventors envisage that this synergy may be used when preparing subjects for haematopoietic stem cell transplants. By replacing irradiation and alkylator treatment and lowering the concentration of fludarabine to be used, this may allow haematopoietic stem cell transplants to be performed in patients for which transplantation was previously not a treatment option owing to the toxicity of high concentrations of fludarabine in such subjects (e.g., aged subjects, subjects suffering from genetic disorders).

Accordingly, one embodiment provides a method of preparing a subject for haematopoietic stem cell transplantation, comprising administering to the subject an effective amount of an immunoconjugate comprising an antibody which specifically binds CD300f, or antigen binding fragment thereof, linked to at least one pyrrolobenzodiazepine dimer, and an effective amount of a lymphodepleting agent. As used herein, a lymphodepleting agent is an agent which depletes lymphocytes when administered to a subject. In one embodiment, the lymphodepleting agent is fludarabine or a pharmaceutically acceptable salt thereof. In one embodiment, the antibody, or antigen binding fragment thereof, comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

In one embodiment, the heavy chain variable region comprises SEQ ID NO: 1, and the light chain variable region comprises SEQ ID NO: 5.

One embodiment provides a pharmaceutical composition for preparing a subject for haematopoietic stem cell transplantation, comprising:
(1) an immunoconjugate comprising an antibody which specifically binds CD300f, or an antigen binding fragment thereof, linked to at least one PBD dimer; and
(2) a lymphodepleting agent.

In one embodiment, the lymphodepleting agent is fludarabine or a pharmaceutically acceptable salt thereof.

In one embodiment, the antibody, or antigen binding fragment thereof, comprises:

(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
   (b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

In one embodiment, the heavy chain variable region comprises SEQ ID NO: 1, and the light chain variable region comprises SEQ ID NO: 5.

The pharmaceutical compositions described herein may be administered by any suitable means, typically, parenterally, such as by subcutaneous, intravenous, intramuscular, intra(trans)dermal, or intracisternal injection or infusion techniques (e.g., as sterile injectable aqueous solutions or suspensions); in dosage unit formulations containing non-toxic, pharmaceutically acceptable vehicles or diluents. The antibody or antigen binding fragment thereof may, for example, be administered in a form suitable for immediate release or extended release. Immediate release or extended release may be achieved by the use of suitable pharmaceutical compositions comprising the compounds, or, particularly in the case of extended release, by the use of devices such as subcutaneous implants or osmotic pumps.

The pharmaceutical compositions for the administration may conveniently be presented in dosage unit form and may be prepared by any of the methods well known in the art of pharmacy. In general, the pharmaceutical compositions are prepared by uniformly and intimately bringing the compound into association with a liquid carrier. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, trihaloacetic (e.g. trifluoroacetic), methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Generally, the term “treating” means affecting a subject, tissue or cell to obtain a desired pharmacological and/or physiological effect and include: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease. In one embodiment, treatment achieves the result of reducing the number of CD300f expressing cells, including AML and/or LSC cells, in the recipient subject.

The term “subject” refers to any animal having a disease which requires treatment by the present method. In addition to primates, such as humans, a variety of other mammals can be treated using the methods of the present invention. For instance, mammals including, but not limited to, cows, sheep, goats, horses, dogs, cats, guinea pigs, rats or other bovine, ovine, equine, canine, feline, rodent or murine species can be treated. Dogs in particular are known to experience multiple myeloma.

The term “therapeutically effective amount” refers to the amount of the immunoconjugate that will elicit the biological or medical response of a tissue, system, animal or human that is being sought by the researcher, veterinarian, medical doctor or other clinician.

In haematopoietic stem cell transplantation, and in the treatment or prevention of AML, an appropriate dosage level will generally be about 0.01 to 50 mg per kg patient body weight per day which can be administered in single or multiple doses. Preferably, the dosage level will be about 0.1 to about 25 mg/kg per day; more preferably about 0.5 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.05 to 0.5, 0.5 to 5 or mg/kg per day.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also disclosed herein is a kit comprising the immunoconjugate described herein, typically comprising one or more containers filled with the immunoconjugate, for the treatment of a condition associated with CD300f expression, such as AML.

In one embodiment, the kit comprises the immunoconjugate, in one or more containers, and one or more other therapeutic agents useful for the treatment of a condition associated with CD300f expression, such as AML, and/or useful for haematopoietic stem cell transplantation.

An used herein, an antibody, or antigen binding fragment thereof, which “specifically binds” an antigen is an antibody, or antigen binding fragment thereof, that reacts or associates more frequently, more rapidly, with greater duration and/or with greater affinity with that antigen than it does with alternative antigens or cells. For example, an antibody, or antigen binding fragment thereof, which specifically binds CD300f is an antibody, or antigen binding fragment thereof, that reacts or associates more frequently, more rapidly, with greater duration and/or with greater affinity with that antigen than it does with alternative antigens or cells. Typically, an antibody, or antigen binding fragment thereof, which specifically binds CD300f, specifically binds to an extracellular domain of CD300f. Such an antibody, or antigen binding fragment thereof, therefore reacts or associates more frequently, more rapidly, with greater duration and/or with greater affinity with an extracellular domain of CD300f than it does with alternative antigens or cells.

An “antibody drug conjugate” (ADC) is an antibody, or antigen binding fragment thereof, that is conjugated to one or more drugs (such as cytotoxins), typically through a linker.

As used herein, the terms “linked” and “coupled” have the same meaning and may be used interchangeable.

As used herein, a “linker” is a molecule which couples an antibody, or antigen binding fragment thereof, to a moiety such as a drug (e.g., a cytotoxin). The linker is typically coupled to the antibody through a cysteine thiol or lysine amine group on the antibody, and is typically formed through reaction of a thiol—reactive group such as a double bond (as in maleimide) or a leaving group such as a chloro, bromo or iodo or an R—sulfanyl group or sulfonyl group, or an amine—reactive group such as a carboxyl group or as defined herein. The term “linker” is used in describing the linker in conjugated form, in which one or both of the reactive termini is absent because of the formation of the bonds between the linker and the antibody (or antigen binding fragment thereof) and the cytotoxin.

As used herein, the term “alkyl” and “alkylene” refers to a straight chain or branched saturated hydrocarbon group. Where appropriate, the alkyl or alkylene group may have a specified number of carbon atoms, for example, C₁₋₆ alkyl, or C₁₋₆ alkylene, which includes groups having 1, 2, 3, 4, 5 or 6 carbon atoms in a linear or branched arrangement. Examples of suitable alkylene groups include, but are not limited to, methylene, ethylene, n-propylene, i-propylene, n-butylene, i-butylene, t-butylene, n-pentylene, 2-methylbutylene, 3-methylbutylene, 4-methylbutylene, n-hexylene, 2-methylpentylene, 3-methylpentylene, 4-methylpentylene, 5-methylpentylene, 2-ethylbutylene, 3-ethylbutylene, heptylene, octylene, nonylene and decylene. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

As used herein, the term “alkenylene” refers to a straight-chain or branched hydrocarbon group having one or more double bonds between carbon atoms and having 2 to 10 carbon atoms. Where appropriate, the alkenylene group may have a specified number of carbon atoms. For example, C₂-C₆ as in “C₂-C₆alkenylene” includes groups having 2, 3, 4, 5 or 6 carbon atoms in a linear or branched arrangement. Examples of alkenylene groups include, but are not limited to, ethenylene, propenylene, isopropenylene, butenylene, butadienylene, pentenylene, pentadienylene, hexenylene, hexadienylene, heptenylene, octenylene, nonenylene and decenylene.

As used herein, the term “alkynylene” refers to a straight-chain or branched hydrocarbon group having one or more triple bonds and having 2 to 10 carbon atoms. Where appropriate, the alkynylene group may have a specified number of carbon atoms. For example, C₂-C₆ as in “C₂-C₆alkynylene” includes groups having 2, 3, 4, 5 or 6 carbon atoms in a linear or branched arrangement. Examples of suitable alkynylene groups include, but are not limited to ethynylene, propynylene, butynylene, pentynylene and hexynylene.

As used herein, the term “cycloalkylene” refers to a saturated cyclic hydrocarbon. The cycloalkylene ring may include a specified number of carbon atoms. For example, a 3 to 8 membered cycloalkylene group includes 3, 4, 5, 6, 7 or 8 carbon atoms. Examples of suitable cycloalkylene groups include, but are not limited to, cyclopropylene, cyclobutylene, cyclopentylene, cyclohexylene, cycloheptylene and cyclooctylene.

As used herein, the term “arylene” is intended to mean any stable, monocyclic, bicyclic or tricyclic carbon ring system of up to 7 atoms in each ring, wherein at least one ring is aromatic. Examples of such aryl groups include, but are not limited to, phenylene, naphthylene, tetrahydronaphthylene, indanylene, fluorenylene, phenanthrenylene, biphenylene and binaphthylene.

The term “heteroalkylene” or “heteroalkynylene” or “heterocycloalkylene” as used herein, refers to a hydrocarbon in which at least one carbon atom has been replaced by heteroatoms independently selected from the group consisting of N, N(R), S, S(O), S(O)₂ and O.

The term “heteroarylene” as used herein, represents a stable monocyclic, bicyclic or tricyclic ring of up to 7 atoms in each ring, wherein at least one ring is aromatic and at least one ring contains from 1 to 4 heteroatoms selected from the group consisting of O, N and S. It is to be understood that the terminology used herein is for the purpose of describing particular embodiments only and is not intended to limit the scope of the present invention which will be limited only by the appended claims. As used herein and in the appended claims, the singular forms “a”, “an”, and “the” include plural reference unless the context clearly indicates otherwise. Thus, for example, a reference to “an antibody” includes a plurality of such antibodies. Unless defined otherwise, all technical and scientific terms used herein have the same meaning as commonly understood by one of ordinary skill in the art to which this invention belongs.

Although any materials and methods similar or equivalent to those described herein can be used to practice or test the present invention, preferred materials and methods are described herein.

All publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

The present application claims priority from Australian application no. 2019902714, the entirety of which is incorporated herein by reference.

In order to exemplify the nature of the present invention such that it may be more clearly understood, the following non-limiting examples are provided.

### EXAMPLES

Haematopoietic stem cell transplantation (HSCT) has been used in the treatment of a number of conditions including Acute myeloid leukemia, Myelodysplastic syndrome, Myeloproliferative neoplasms, Chronic myeloid leukemia, Chronic myelomonocytic leukemia, Bone marrow failure syndromes, Sickle cell disease, Thalassemia, Primary immunodeficiency disorders, inherited metabolic disorders, and gene modified autologous transplantation.

Treatment related mortality (TRM) in haematopoietic stem cell transplantation remains significant, especially in those above the age of 65. The development of antibody-based therapies depleting hematopoietic stem and progenitor cells (HSPC) as part of HSCT conditioning may reduce or eliminate traditional methods of depleting HSPC such as alkylating agents and irradiation.

CD300f is an inhibitory receptor found on healthy myeloid antigen presenting cells (APC) (Borrego F. Blood. 2013; 121(11):1951-1960; Clark et al. Trends Immunol. 2009; 30(5):209-217). CD300f is present on a high proportion of AML cells as well as HSPC (Korver et al. Leukemia. 2009; 23(9):1587-1597; Abadir et al. Mol Oncol. 2019; 13(10):2107-2120). We have demonstrated how incorporating an anti-CD300f ADC into conditioning for allogeneic HSCT may decrease relapse and toxicity by reducing and/or replacing traditional non-specific toxic agents.

### Materials and Methods

Preparation of Tissue Samples

Blood and bone marrow (BM) samples from patients with AML were collected at Concord Repatriation General Hospital (CRGH) or Royal Prince Alfred (RPA) Hospital (Sydney Australia). Peripheral blood (PB) or BM samples from healthy donors were collected at CRGH. Cord Blood (CB) samples were collected by the Sydney CB bank. All donors provided informed consent under ethical approval obtained from the Sydney Local District Human Research Ethics Committee (HREC/12/CRGH/59, HREC/11/CRGH/61 & 118). Mononuclear cells (MC) were isolated from samples using density gradient centrifugation through Ficoll-Paque Plus (GE Healthcare) according to the manufacturer's protocols. BM aspirates were collected from healthy participants from the posterior iliac crest. Samples were passed through a 22g needle to disrupt BM fragments and then filtered prior to MC isolation as above. Human monocytes were purified from MC by CD14 Microbeads selection using an AutoMACS Pro (Miltenyi Biotec).

Cell Lines

The AML cell lines HL-60, THP-1 and erythroleukemic cell line, HEL, were obtained from the Christchurch School of Medicine, University of Otago. U973, Z-138 and Mino cell lines were from the American Type Culture Collection (ATCC). All cell lines were maintained in complete RPMI 1640 (complete RPMI) containing 10% FCS, 2 mM Gluta-MAX, 100 U/ml penicillin and 100 μg/mi streptomycin, ThermoFisher).

Gene Expression

AML gene expression data was retrieved from the Gene Expression Omnibus microarray dataset GSE14468 (Wouters et al. Blood. 2009; 113(13):3088-3091). HSPC gene expression was retrieved from GSE42519, GSE17054 and GSE19599 (Rapin et al. Blood. 2014; 123(6):894-904; Majeti et al. Proc Natl Acad Sci USA. 2009; 106(9):3396-3401). The series matrix files were parsed in R and the probe ID and signal values corresponding to CD300LF (1553043_a_at) and CD33 (206120 at) extracted. GTEx data was analysed in Rstudio. Tissue type and transcription data in transcripts per million for CD300LF and CD33 were extracted from all experiments in this data set.

Antibodies and Generation of DCR-2-Pyrrolobenzodiazepine (PBD) and Isotype-PBD

Monoclonal antibody DCR-2 (IgG1, κ) is produced by a hybridoma deposited under the Budapest Treaty at CellBank Australia, 214 Hawkesbury Rd., Westmead, NSW 2145, Australia, on 27 Sep. 2016 and designated accession number CBA20160029. DCR-2 is a murine immunoglobulin G1 antibody that binds all extracellular forms of CD300f.

Antibodies were conjugated to pyrrolobenzodiazepine dimer by native cysteine chemistry via a Cathepsin B-cleavable linker, MA-PEG4-VA-PBD (SET0212—Levena Biopharma, Calif.). Briefly, mAb was reduced with 10 mM dithioerythritol to expose free thiols of interchain disulfides and purified by PD-10 column. The reduced antibody (2 ml aliquots of antibody) was reacted with a 10-fold molar excess of PBD linker (MA-PEG4-PBD) (10 mg/ml) for 3 hrs. before overnight dialysis into phosphate-buffered saline (PBS). The final drug to antibody Molar ratio was 3.3 for DCR-2-PBD and 4.6 for isotype-PBD.

### Colony Forming Unit Toxicity Assay

CFU colonies were plated at 2×10⁴ cells/well in semi-solid methylcellulose medium (MethoCult Classic, Stemcell Technologies) in a 24 well plate (Corning, N.Y., USA), were cultured at 37° C. in 5% CO₂ for 14 days and counted via microscopy. Whole cord blood PBMC was used. CD34⁺ progenitors were continuously exposed to multiple concentrations of DCR-2 with equal concentrations of a secondary goat anti mouse antibody conjugated to PBD dimer. As a control, only the secondary PBD dimer conjugated antibody was used as above.

### Activation Marker Expression and Monocyte-Derived Dendritic Cells (MoDC) Toxicity Assays

The activity of DCR-2PBD on the upregulation of activation markers on DC was measured by incubating whole PBMC with DCR-2PBD or no antibody for 6 hours before washing, followed by 12 hours of LPS stimulation. Control DC were not exposed to DCR-2PBD or LPS. Myeloid DC were gated out by excluding dead cells, Lin⁺(CD3, CD20, CD19, CD56, CD14), then gating on HLA-DR⁺ and CD11c⁺ cells. The activation markers CD80 and CD83 were assessed via flow cytometry. The changes on activation were determined by comparing activated DC incubated with or without DCR-2PBD compared to inactivated DC.

DCR-2PBD's impact on antigen presentation was assessed by incubating DCR-2PBD or isotope-PBD with whole PBMC for 24 hours then washing, afterwards adding allogenic CFSE labeled T cells (naive CD4, magnetically separated) and measuring T cell division via reduction in CFSE expression. In more detail, T cells were depleted from PBMC by magnetic selection using an AutoMACS Pro with anti-CD3 mAb (HIT3a, Biolegend) with greater than 90% depletion in all samples. The CD3-depleted PBMC were incubated in complete RPMI media with DCR-2-PBD (200 pmol), isotype-PBD (200 pmol) or PBS for 24 hours. After washing to remove unbound ADC, the CD3-depleted PBMC were used to stimulate allogeneic CFSE labelled naive CD4+ T cells which had been prepared using a RosetteSep Kit (Stem Cell Technologies 17555). On day 5 the proliferation of T cells, identified using anti-CD3 AF700 (SP34-2), was assessed by CFSE reduction using flow cytometry. The results of the DCR-2-PBD and isotype-PBD groups were normalised to the PBS control group. Stimulator populations were prepared from three PBMC donors and experiments were performed in duplicate.

Internalisation Assays

DCR-2 (IgG₁), and as an isotype control, the anti-tetanus toxoid mAb, CMRF-81 (IgG₁) which were both produced, purified and directly conjugated to phycoerythrin (PE) in house (Abadir et al. Mol Oncol. 2019; 13(10):2107-2120). Cells were incubated with DCR-2-PE or CMRF-81-PE (10 μg/ml) on ice for 20 min, washed to remove unbound mAb then incubated at 37° C./5% CO₂ to allow internalisation for the indicated times.

After incubation, residual mAb remaining on the cell surface was detected with a secondary goat anti-mouse (GAM) IgG-AF488 antibody. Cells were fixed in 1% paraformaldehyde/PBS for flow cytometry analysis. The surface and total relative Mean Fluorescent Index (MFI) was calculated as the (MFI of binding antibody at time point−MFI of isotype control at time point) at 37° C./(MFI of binding antibody at time point−MFI of isotype control at time point) at 0° C. The percent internalised was assessed by 100−(relative MFI surface staining/relative MFI total staining). In immunofluorescent microscopy experiments cells were stained with DCR-2 then air-dried and fixed with 4% paraformaldehyde or incubated for 37° C. for 30 minutes then fixed. Rehydration was performed using 1% BSA/PBS prior to staining with GAM IgG-AF488 antibody and 18 μM DAPI (ThermoFisher).

Cytotoxicity Assays

Cytotoxicity was determined by incubating 5000 AML or lymphoma cells with DCR-2-PBD, isotype-PBD or PBS control in 200 μl total volume of complete RPMI for 96 hours at 37° C./5% CO₂ after which DAPI⁻viable cells were enumerated using flow cytometry. Events per condition were compared to the mean of the control group to obtain the % viable. Bystander killing of CD300f⁻ Mino cells was performed as above. After incubation live bystander cells were identified with ant-CD20-PE antibody (2H7) and DAPI. Kinetic analysis was performed as above. At the indicated time points cells were washed 3 times then resuspended in complete RPMI and cultured for 96 hours before analysis. Synergy between ADC and fludarabine was assessed as above by combining DCR-2-PBD and/or fludarabine in samples. The Combination Index (Cl) was calculated using Compusyn software (Hsu et al. Oncolmmunology. 2018; 7(4): e1419114).

Activation Marker Expression and Monocyte-Derived Dendritic Cells (MoDC) Toxicity Assays

PBMC were incubated in complete RPMI media with or without DCR-2-PBD (200 pmol). After 12 hours, washed cells were incubated for a further 12 hours with or without LPS then phenotyped for the expression of activation markers on DC populations. MoDC were derived from CD14+ monocytes as described previously (Fromm et al. J Leukoc Biol. 2020; 107(2):323-339). DCR-2-PBD or isotype-PBD (200 pmol) were added for 72 hours prior to enumeration of live cells by flow cytometry.

Mixed Leucocyte Reaction

T cells were depleted from PBMC by magnetic selection using an AutoMACS Pro with anti-CD3 mAb (HIT3a, Biolegend) with greater than 90% depletion in all samples. The CD3-depleted PBMC were incubated in complete RPMI media with DCR-2-PBD (200 pmol), isotype-PBD (200 pmol) or PBS for 24 hours. After washing to remove unbound ADC, the CD3-depleted PBMC were used to stimulate allogeneic CFSE labelled naive CD4+ T cells which had been prepared using a RosetteSep Kit (Stem Cell Technologies 17555). On day 5 the proliferation of T cells, identified using anti-CD3 AF700 (SP34-2), was assessed by CFSE reduction using flow cytometry. The results of the DCR-2-PBD and isotype-PBD groups were normalised to the PBS control group. Stimulator populations were prepared from three PBMC donors and experiments were performed in duplicate.

Colony Forming Unit Toxicity Assay

CB cells, plated at 2×10⁴ cells/well in semi-solid methylcellulose medium (MethoCult Classic, Stemcell Technologies) in a 24 well plate (Corning, N.Y., USA), were cultured at 37° C. in 5% CO₂ for 14 days when colony forming units (CFU) were scored. DCR-2-PBD or isotype-PBD was added to each well for continuous exposure. Three CB samples were tested with duplicate wells performed in each experiment.

Mouse Xenograft Assays

NOD.Cg-Prkdc^{scld}//2rg^{tm1Wjl}/SzJ (NSG) mice obtained from Australian BioResources were housed under specific pathogen free conditions. In the subcutaneous model, 2×10⁶ U937 cells were injected under the skin on the right flank. Tumours were measured daily with electronic calipers. When the mean volume of the tumours was >100 mm³, DCR-2-PBD, isotype-PBD or PBS was injected intraperitoneally once. Mice were assigned so each condition had a similar mean tumour volume (range 130.3-137.2 mm³). Tumour volume was measured until >1000 mm³ when mice were euthanised as required by the ethics protocol.

For the HL-60 leukemic model, NSG mice were irradiated with 200cGy 24 hours prior to intravenous administration of 5×10⁶ HL-60 cells. On day 7, mice were injected with DCR-2-PBD, isotype-PBD or PBS and monitored for disease progression and survival. Mice were euthanised when their clinical score was ≥4 or on d70.

The humanised mouse model used frozen CD34⁺ cells (>95% purity) isolated by positive selection (Miltenyi 130-046-702) according to the manufacturer's instructions from two pooled CB samples. Each NSG mouse was injected with 100,000 CD34+ cells 4 hours after receiving a 150cGy irradiation dose. Engraftment as determined by % human CD45+ cells in venous blood was assessed at 12 weeks. Mice were assigned to treatment groups to have similar means of human CD45+ percentage in the PB (10.16% DCR-2-PBD cohort and 10.74% isotype-PBD cohort). Mice were then injected with 300 ug/kg of DCR-2-PBD or isotype-PBD. Mice were euthanised on day 7 and human CD45+ cells from BM (bilateral femurs and tibias) and 300 μl blood were enumerated and phenotyped by flow cytometry.

For primary AML xenografts, NSG mice were irradiated with 150cGy 24 hours before 8×10⁶ AML cells from CRGH11 (sample 10 Table S1) were injected iv. Once engraftment was established (>1% human CD45 positive events in PB), mice were given DCR-2-PBD or isotype-PBD. Mice were euthanised 6 days later and the BM was harvested for enumeration and surface marker analysis by flow cytometry.

In the flank injection model 2×10⁶ U937 cells were injected subcutaneously into NSG mice and the subsequent tumors were measured daily via caliper to calculate the volume. Mice were injected with 150 ug/kg of DCR-2ADC, Isotype-ADC or an equal volume of PBS on day 6. Mice were euthanized once the volumes reached 1000 mm³. Survival and tumor size was recorded.

Flow Cytometry

CD300f expression on primary AML and BM samples was performed on an Influx flow cytometer (BD Biosciences). Mouse AML cell line experiments were performed on an Accuri flow cytometer (BD Biosciences). Remaining assays were performed on a Fortessa LSR flow cytometer (BD Biosciences). Analysis, including TiSNE, was performed using FlowJo (FlowJo LLC).

Statistical Analysis

Statistical analysis was performed using Prism (GraphPad Software, Inc.). Error bars correspond to standard error. Differences in means between two groups were assessed using t tests. Multiple group tests were performed using ANNOVA with post-test comparisons. Differences in survival was assessed using a Logrank (Mantel-Cox) test.

Results

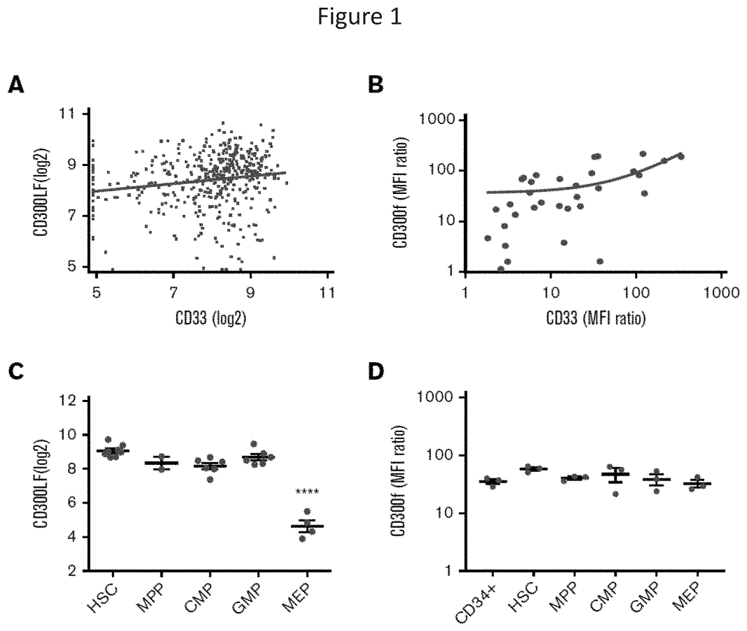

To assess the distribution of CD300f, gene expression data were analyzed. As gentuzumab ozogamicin, which targets CD33, is the only approved AML therapeutic that binds a surface molecule, expression of CD300f was compared to CD33 in AML. Expression array data was analysed from patients with AML (n=460) to compare CD33 and CD300 (FIG. 1A). The mean CD300f expression level was significantly higher than CD33 (8.39 log 2 vs 7.89 log 2, P<0.0001). There were no significant differences in CD300 expression across the European LeukemiaNet risk groups.

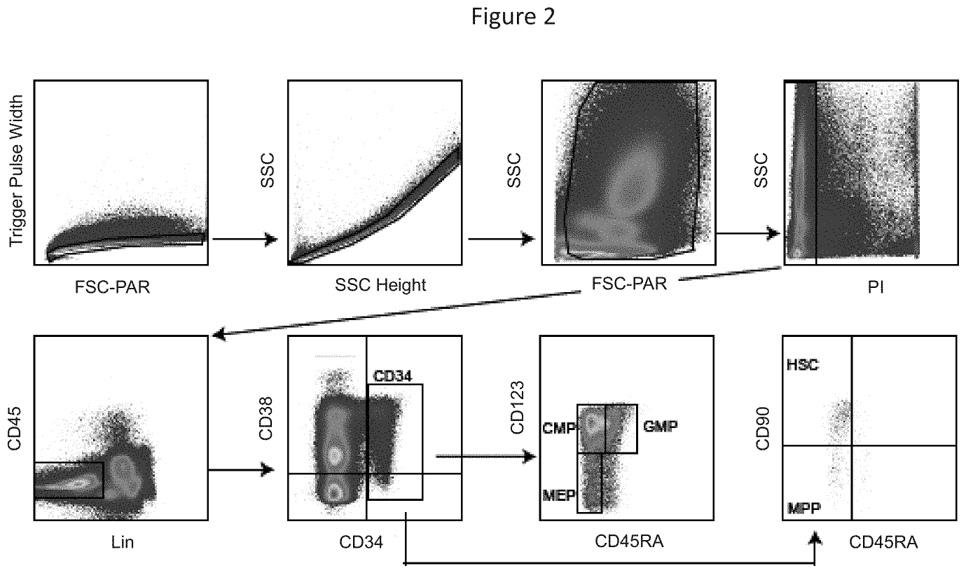

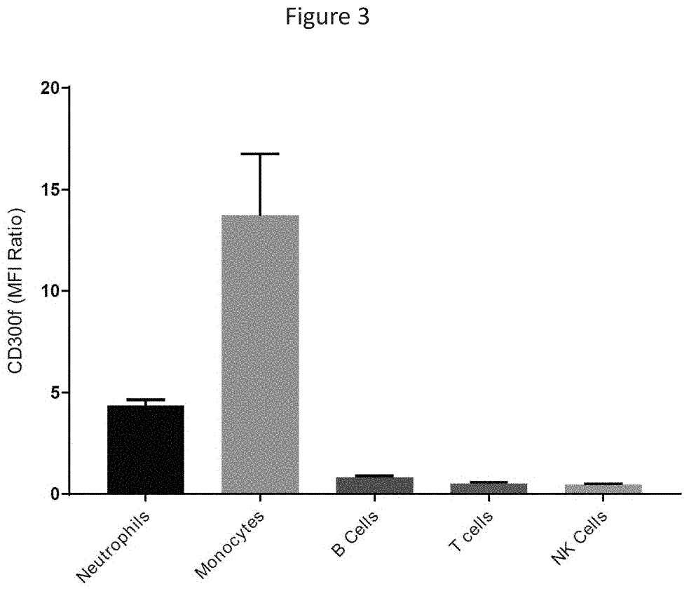

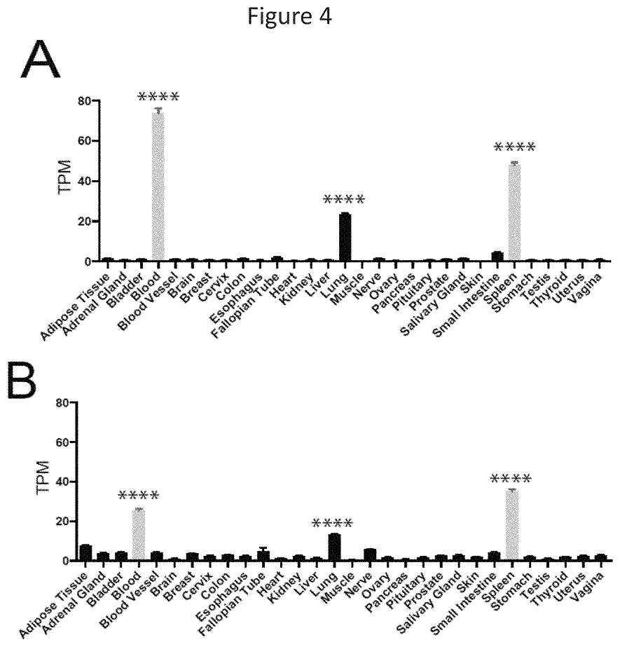

Protein expression of CD300f and CD33 was compared by flow cytometry (FIG. 1B). There was no significant difference in mean fluorescence intensity (MFI) ratios, and there was a moderate to strong correlation in expression (r=0.63, P≤0.0001). CD300f protein expression on the CD34+CD38− subset of primary AML showed no significant correlation with CD33 protein expression. Single-cell expression was compared on a set of 9 AMLs using TiSNE, with a similar distribution seen for CD300f and CD33. Gene expression array data demonstrated CD300f expression across hematopoietic stem cells and myeloid hematopoietic progenitor cells (FIG. 1C), with the lowest expression in megakaryocyte/erythroid progenitors (P<0.0001). Protein expression by flow cytometry did not reveal any significant differences in MFI ratio across the groups of HSPCs. A standard gating strategy (FIG. 2) was used to determine HSPC subsets. Protein expression on peripheral blood mononuclear cells showed CD300f expression on monocytes and neutrophils, but not lymphocytes (FIG. 3). RNA sequencing (RNA-seq) data from the human GTx database of multiple organs were analyzed for potential off-target effects of anti-CD300f-based therapy. Both CD33 and CD300LF had significantly increased expression in the blood, spleen, and lungs compared with all other organs (P<0.0001) (FIG. 4). Immunohistochemistry analysis by The Human Protein Atlas (https://www.proteinatlas.org/) indicates that CD33 and CD300f are expressed on lung macrophages, but not on pneumocytes.

Antibodies to CD300f are Internalised Upon Binding

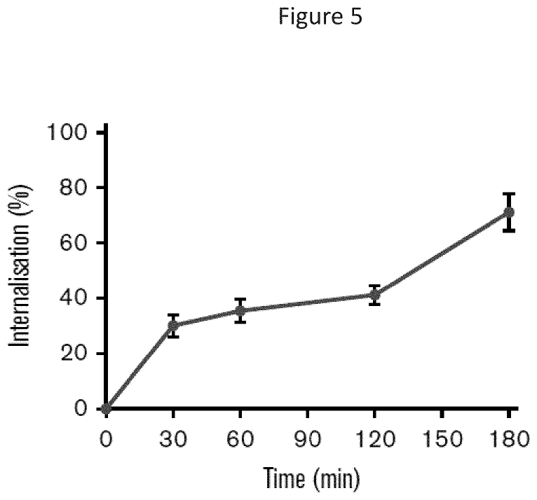

The mouse anti-human CD300f antibody, DCR-2 was assessed for its ability to be internalised by flow cytometry. It rapidly internalises upon binding to HL-60 cells (FIG. 5). Internalisation is significant at 30 minutes and is greater than 50% at 180 minutes. Internalisation at 30 minutes was confirmed by fluorescence microscopy on HL-60, CD14+ monocytes and primary AML cells. The internalisation data indicates that DCR-2 is a suitable mAb for ADC development.

In Vitro Cytotoxicity of DCR-2-PBD

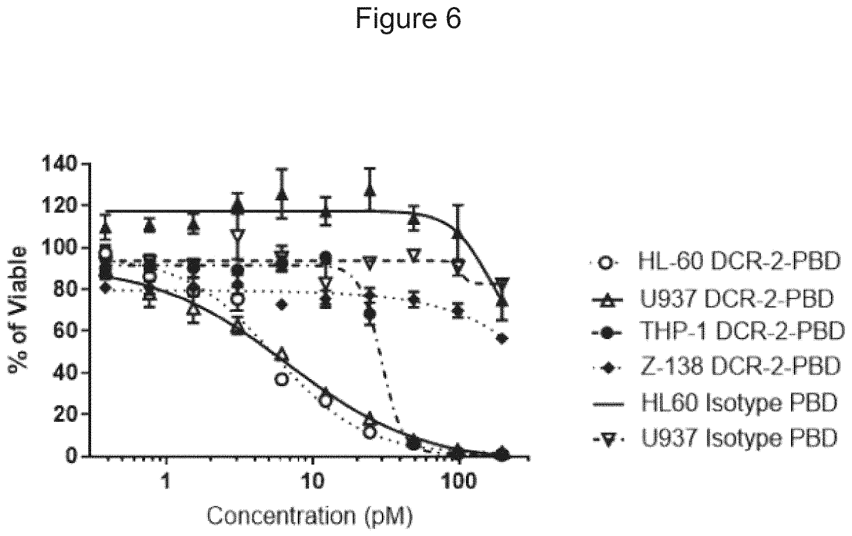

DCR-2 and an isotype control mAb were conjugated to a PBD dimer toxin, to assess potential cytotoxicity. The cytotoxicity of DCR-2-PBD and the isotype-PBD were tested against CD300f+ and CD300f− cell lines and the results are shown in FIG. 6 and Table 2.

TABLE 2

| | IC50 (pM) | | | | | | |
|---|---|---|---|---|---|---|---|
| | | | IMGN779 | SGN-33a | SGN-123a | | |
| | DCR- | Gemtuzumab- | (CD33 | (CD33 | (CD123 | | |
| Cell Line | 2PBD | Ozogamicin | ADC) | ADC) | ADC) | Doxorubicin | Busulfan |
| HL-60 | 5.448 | 13.3 | 2.8 | 6.667 | | 345 × 10⁶ | 354 × 10⁶ |
| U937 | 6.738 | 126.7 | | 146.674 | | | |
| THP-1 | 29.93 | 40 | | 46.669 | 160.008 | | |
| HEL | 544.3 | | 500 | | | | |

DCR-2-PBD killed the CD300f⁺ AML cell lines HL-60, U937 and THP-1 with IC50s in the low picomolar range (5.44 pM, 6.74 pM and 29.39 pM respectively). These IC50s are similar to those of other PBD based ADC (Chou T C. Cancer Res. 2010; 70(2):440-446; Li et al. Mol Cancer Ther. 2018; 17(2):554-564). The IC50s of DCR-2-PBD and the isotype-PBD on the CD300⁻ lymphoma cell line Z-138 were >200 pm. The isotype-PBD has an IC50 of 0.200 pM against HL-60 and U937.

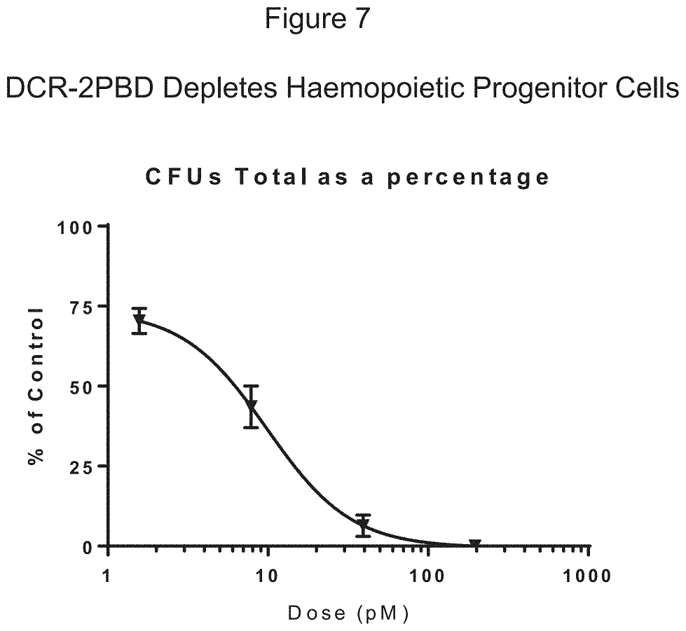

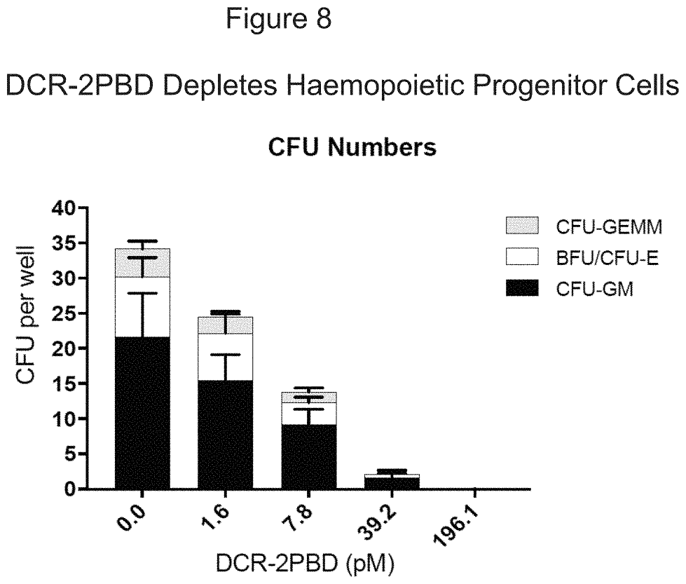

We used a CFU assay to test DCR-2-PBD activity against HSPC and primary AML. Significant reductions in total CFUs was seen at 7.84 pM (p=0.0033), 39.21 pM (p<0.0001) and 196.1 pM (p<0.0001) (FIG. 7). All major CFU subtypes were inhibited at the 32.9 pM and 196.1 pM concentrations by DCR-2-PBD (FIG. 8). Therefore, DCR-2PBD effectively depletes haematopoietic progenitor cells in a dose dependent manner. DCR-2PBD is effective at depleting multilineage, erythroid and myeloid progenitor cells in a dose dependent manner.

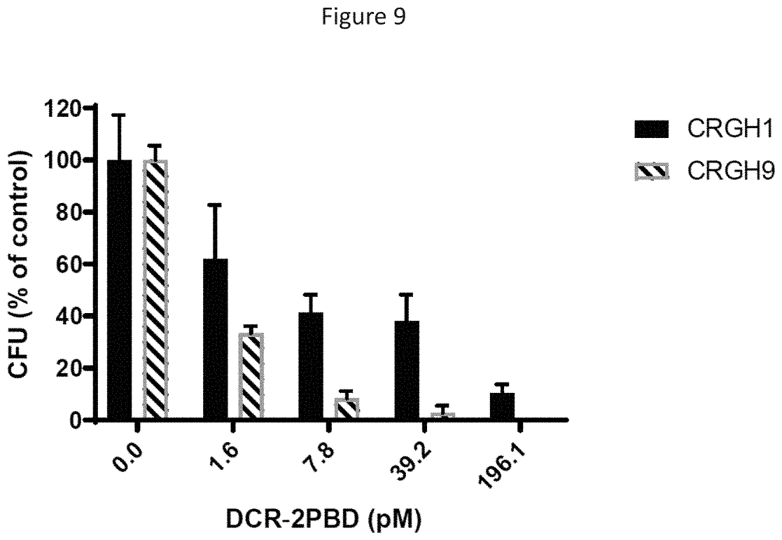

Two primary AML samples were tested: CRGH1 with low CD300f expression (MFI ratio of 2.6) and CRGH9 with a high CD300f expression (MFI 109.9) (FIG. 9). Significant toxicity was demonstrated against CRGH1 at 196.1 pM (P 5.036). Significant toxicity was seen in CRGH9 at all dose levels (P<0.001). This indicates that DCR-2PBD is also effective at depleting AML cells.

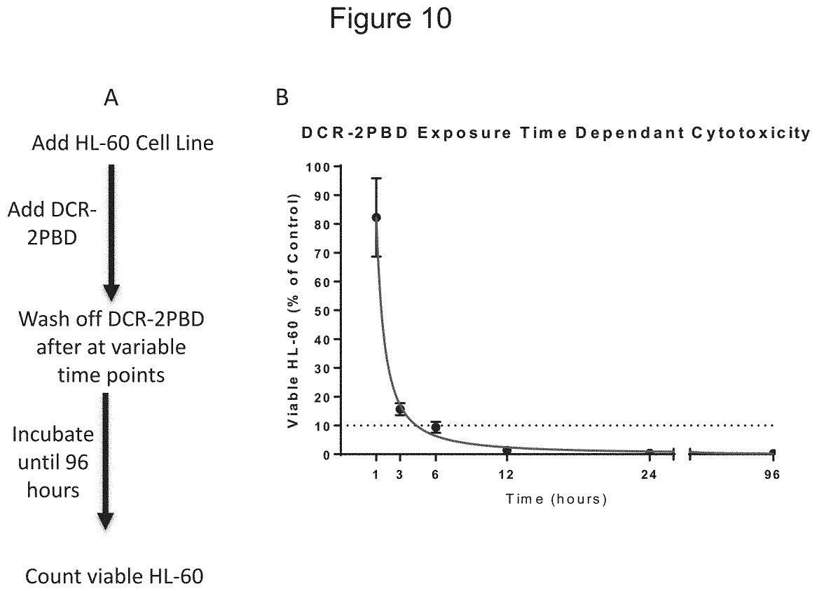

The exposure time required for DCR-2-PBD to cause cytotoxicity on the HL-60 cell line was assessed and the results are shown in FIG. 10. The viability of HL-60 cells was reduced to less than 10% within 5 hours of exposure to DCR-2PBD. At 12 hours, greater than 98% of HL-60 proliferation was inhibited while at 24 and 96 hours inhibition was greater than 99%.

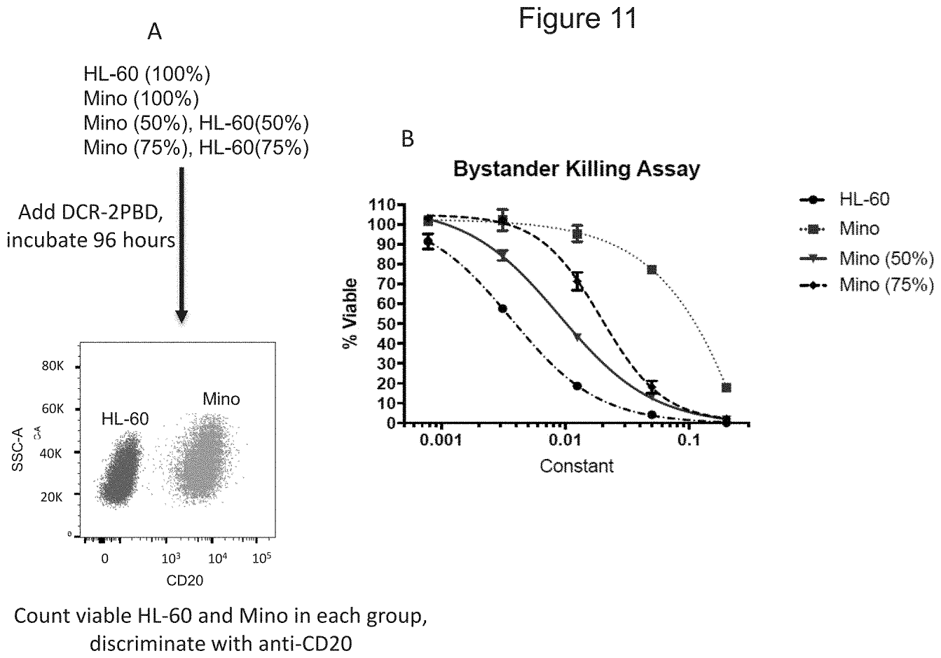

AML surface targets, including CD300f, can be heterogeneously expressed within AML cases and we considered bystander killing advantageous for an effective ADC. To determine whether DCR-2PBD effects cells not expressing CD300f, we investigated bystander killing by comparing the viability of the CD300f− lymphoma cell line Mino cultured on its own, with Mino cultured mixed with HL-60 in the presence of DCR-2-PBD in mixtures of 50% HL-60/50% Mino, and 25% HL-60/75% Mino. Cells were incubated for 96 hours with various concentrations of DCR-2PBD (FIG. 11). There was a significant reduction in viability in the 75% Mino condition with DCR-2-PBD at 25 pM (p=0.0002) compared to the Mino only control (FIG. 10). In the 50% Mino condition there was a significant reduction in viability in both the 25 pM (p<0.0001) and the 12.5 pM (p=0.0004) DCR-2-PBD concentrations (FIG. 11). The reduction in the Mino cells when combined with HL-60 confirm the ability of DCR-2-PBD to generate bystander killing.

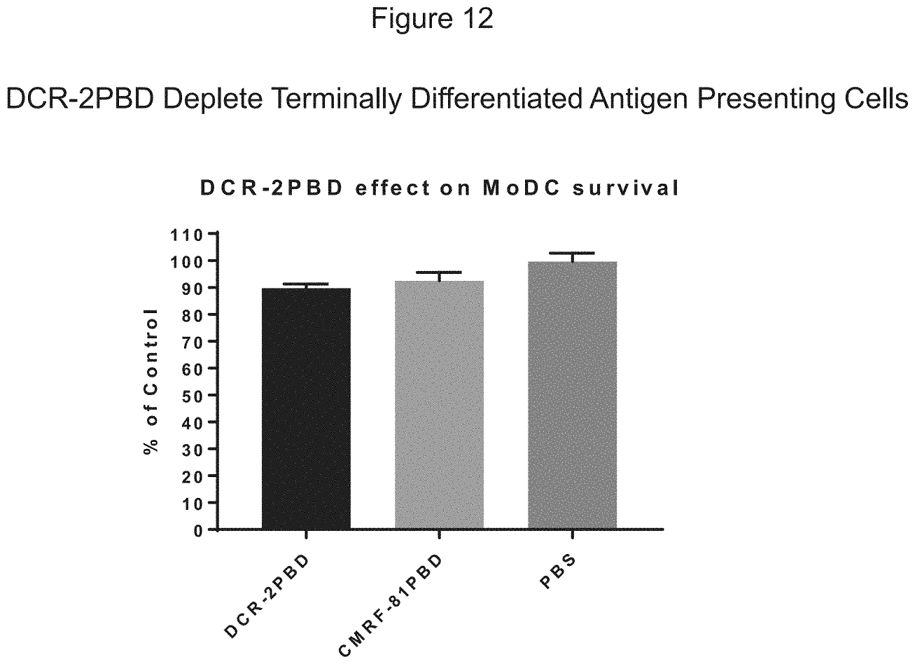

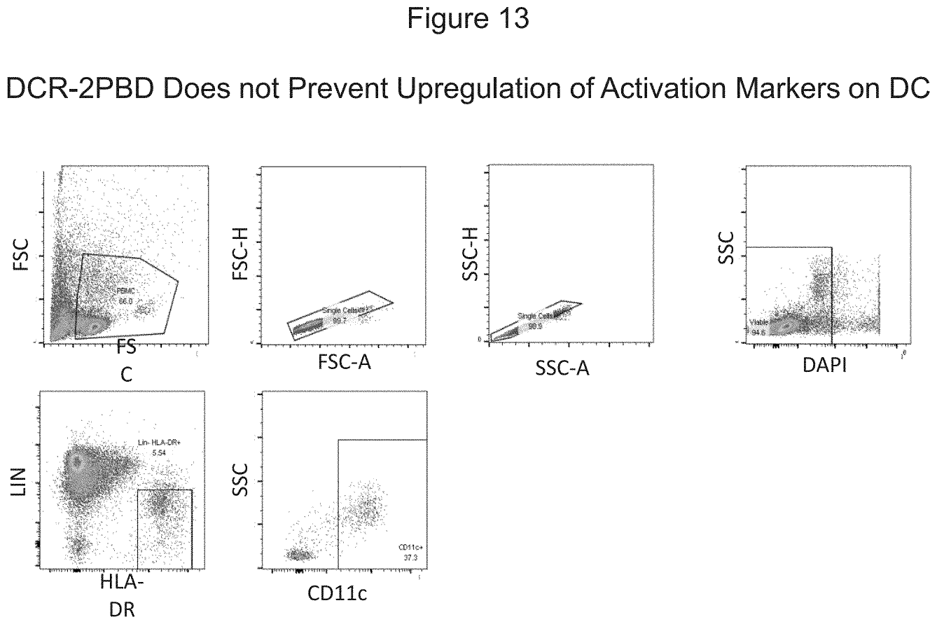

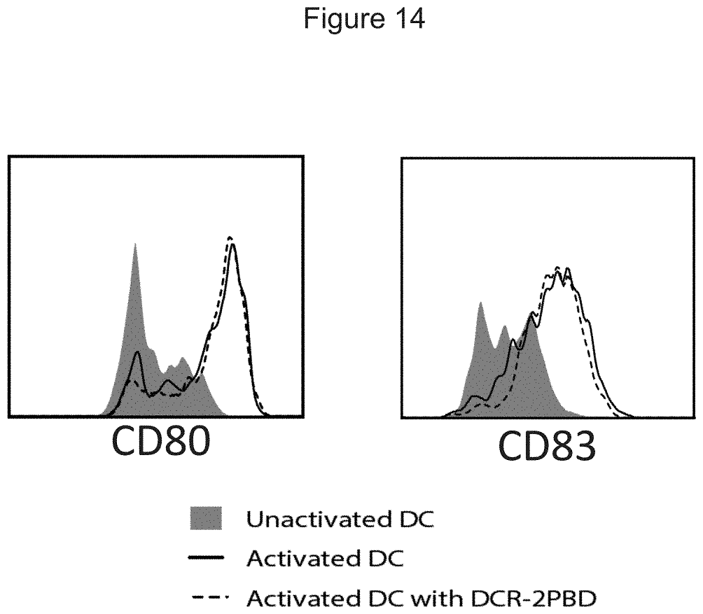

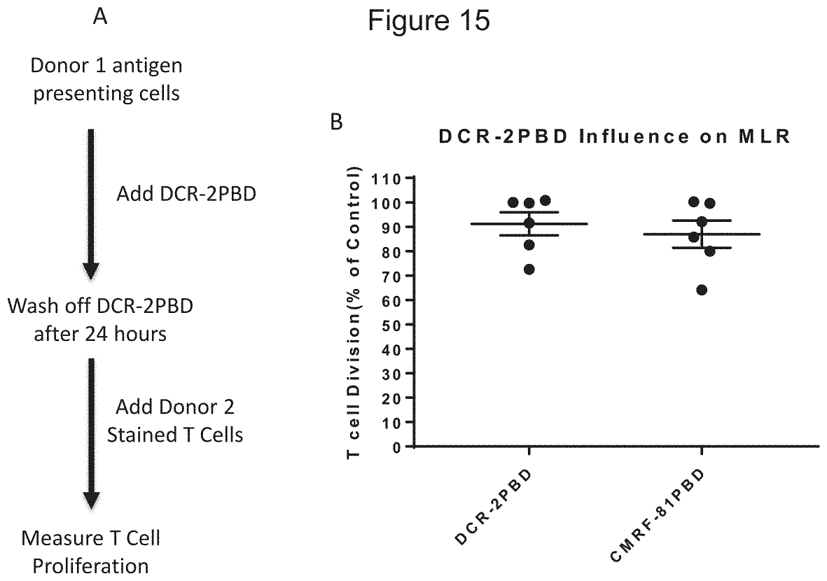

The effect of DCR-2PBD on terminally differentiated antigen presenting cells (monocyte-derived dendritic cells (MoDC)) was compared to the effect of CMRF-81PBD and PBS. The results are shown in FIG. 12. As can be seen from FIG. 12, DCR-2PBD had little effect on MoDC cells. This suggests that DCR-2PBD has little impact on antigen presentation. Further, treatment of dendritic cells with DCR-2PBD, followed by treatment of the dendritic cells with LPS, resulted in upregulation of dendritic cell activation markers HLA-DR, CD11c (FIG. 13), CD80 and CD83 (FIG. 14), illustrating that DCR-2PBD does not prevent up-regulation of activation markers on dendritic cells. Further, FIG. 15 shows that DCR-2PBD has no impact on T cell proliferation in a mixed lymphocyte reaction.

### DCR-2-PBD Prolongs the Survival of Mice AML Cell Line Models

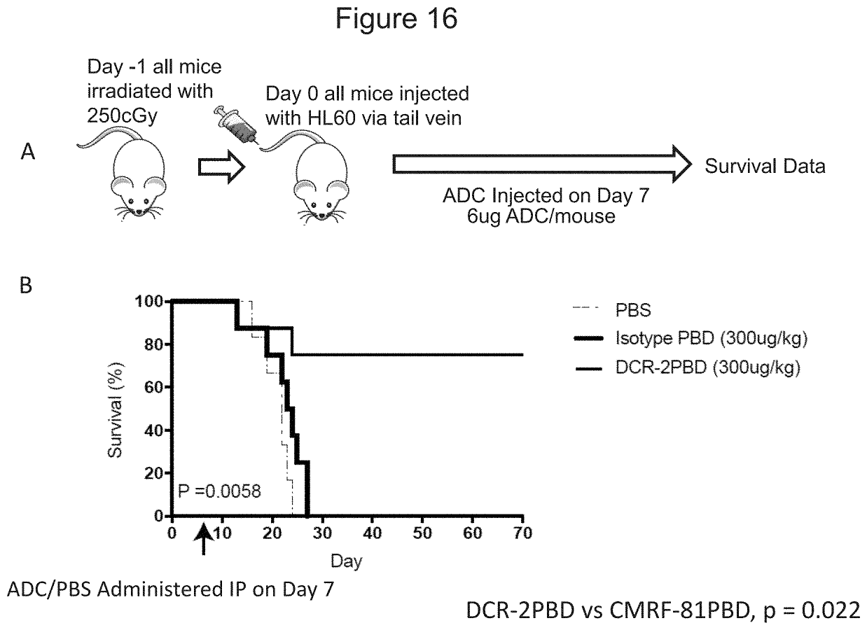

To determine the effect of DCR-2PBD on an AML mouse model, mice were irradiated with 250 cGy, and injected intravenously with HL-60 as shown in FIG. 16, which lead to heavy BM infiltration, meeting clinical endpoints at a median of day 23. On day 7, mice received a single injection of either PBS, or 300 μg/kg of CMRF-81PBD or DCR-2PBD, and their survival monitored. The survival data is shown in FIG. 16.

A single injection of DCR-2-PBD (300 ug/kg) significantly increased survival (p=0.0058) compared to isotype-PBD (FIG. 16). Six of 8 animals treated with DCR-2-PBD survived to the experiment endpoint. No human cells could be detected in mice surviving until day 70, and the range of human CD45+ cells in all other mice was 76% to 88% at the time of euthanasia.

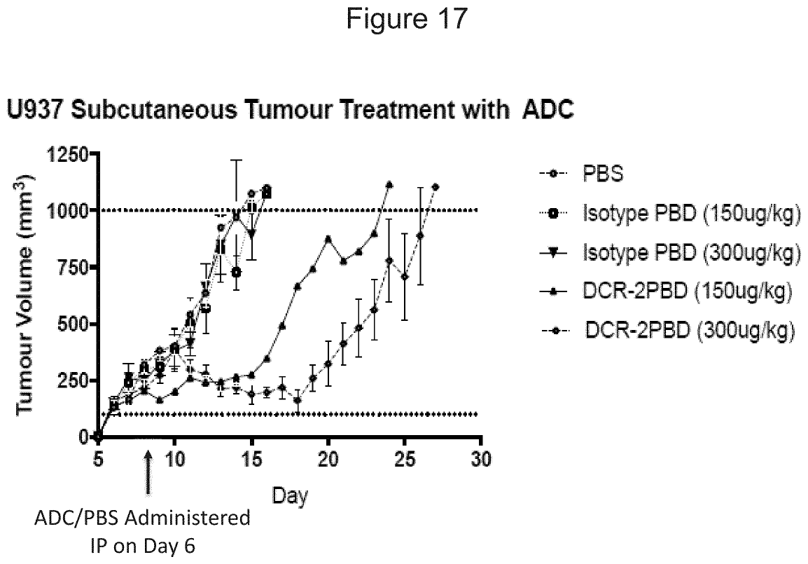

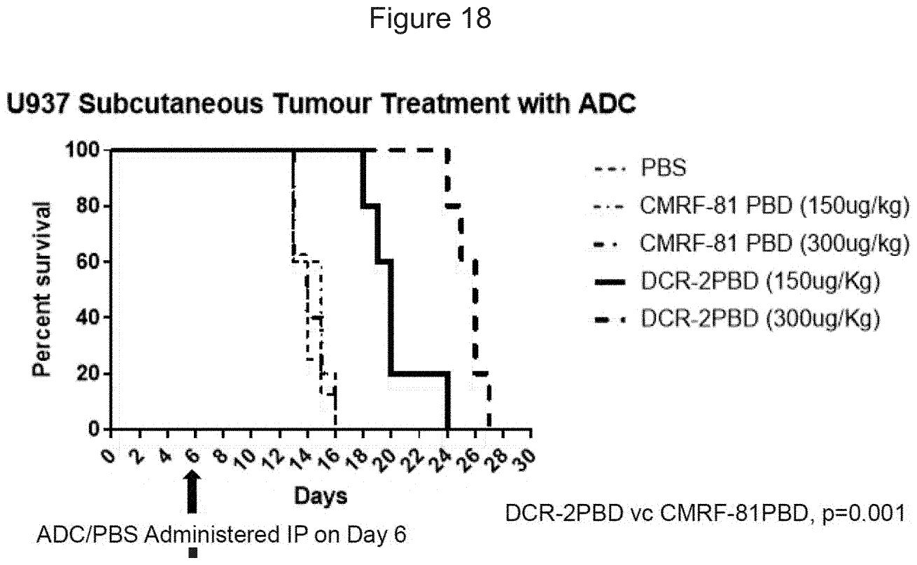

To determine the effect of DCR-2PBD in a mouse model based on U937 cells, mice were injected subcutaneously with cell line U937. After 6 days, mice were administered PBS, 150 mg/kg or 300 mg/kg of CMRF-81PBD or 150 mg/kg or 300 mg/kg of DCR-2PBD intraperitoneally and monitored for tumour growth. Tumour volume and survival of the mice were assessed and the results are shown in FIGS. 17 and 18, respectively. Tumour growth was delayed, and survival was significantly increased in both the 150 ug/kg group (p=0.0017) and 300 ug/kg (p=0.0017) of DCR-2-PBD compared to the isotype-PBD (300 ug/kg) group.

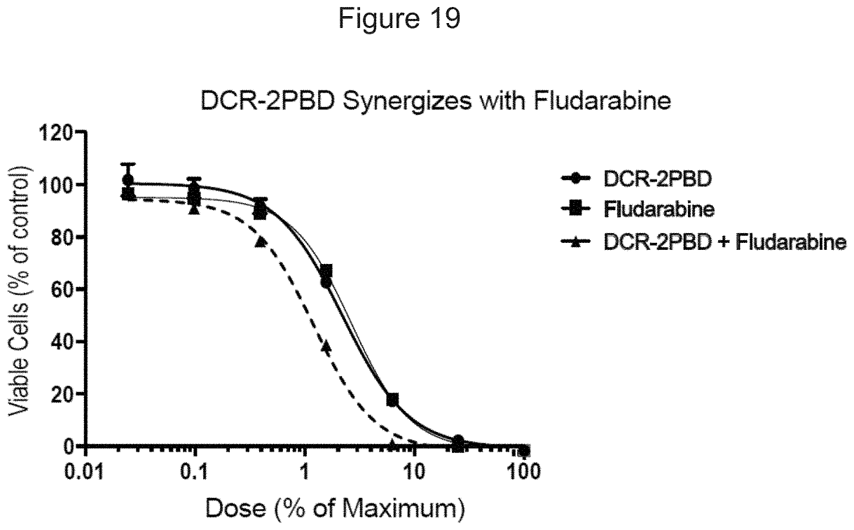

Fludarabine is used in multiple allo-HSCT regimens to facilitate donor cell engraftment by depleting host lymphocytes. An anti-CD300f therapeutic would deplete recipient HSPC but not host lymphocytes as part of a conditioning regimen. Given these complementary roles, we assessed a synergistic relationship between DCR-2-PBD and fludarabine. To determine the effect of a combination of DCR-2PBD and fludarabine on HL-60 cells, HL-60 cells were incubated in vitro with various concentrations of DCR-2PBD alone, with fludarabine alone, or with a mixture of DCR-2PBD and fludarabine. After 96 hours, viable cells were counted, and the results plotted (FIG. 19). The same experiment was also carried out with THP-1 cells.

Cytotoxicity of HL-60 and THP-1 was greater in the presence of both DCR-2-PBD and fludarabine than either alone confirming a synergistic relationship with a Cl of 0.82 and 0.76 respectively.

This means that, when fludarabine is used in combination with DCR-2PBD, a similar cytotoxic effect can be achieved using a lower concentration of fludarabine. The inventors envisage this may permit subjects requiring haematopoietic stem cell transplants who previously were unable to tolerate fludarabine treatment to now undergo a combination treatment with a lower dose of fludarabine.

### DCR-2-PBD Depleted Both Primary HSPC and AML Cells in Mouse Xenografts

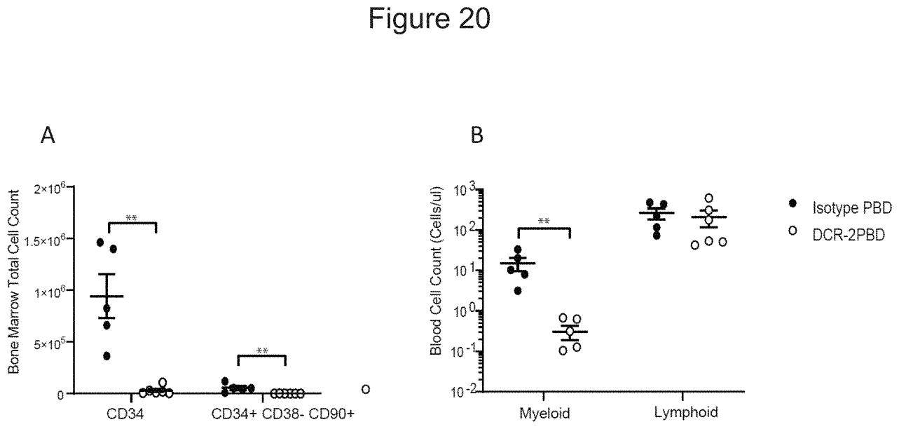

To further show that anti-CD300f ADC could be effective in HSCT conditioning was demonstrated by using DCR-2-PBD to selectively deplete HSPC and myeloid cells in a healthy CD34⁺ engrafted mouse model. Humanised mice injected with a single dose of DCR-2-PBD (300 ug/kg) had a significant depletion in the mean of both CD34⁺ (0.29×10⁵vs 9.42×10⁵, p=0.001) and primitive CD34⁺CD38⁻CD90⁺ cells (0.05×10⁴ vs 5.54×104, p=0.008) compared to isotype-PBD treated groups (FIG. 20A). The difference was significant as a percentage of total human CD45⁺ cells for both the CD34⁺ (3.09% vs 20.62%, p<0.0001) and CD34⁺CD38⁻CD90⁺ populations (0.16% vs 2.17%, p=0.008). The selectivity of DCR-2-PBD was demonstrated by depleting the myeloid cells in the peripheral blood (0.31 cells/μl vs 14.89 cells/μl, p=0.0002) but not lymphocytes (208.5 cells/μl vs 263 cells/μl) which are longer lived and do not express CD300f (FIG. 20B).

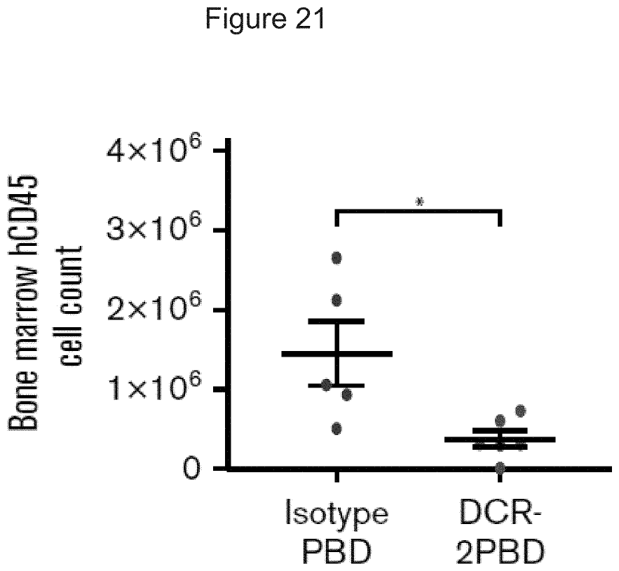

We demonstrated the efficacy of DCR-2-PBD against primary AML by injecting mice with a single dose of 300 ug/kg DCR-2-PBD or isotype-PBD and enumerating BM engraftment on Day 7 (FIG. 21). DCR-2-PBD significantly depleted the human AML cells residing in the BM compared to isotype control when measured by mean total enumeration (0.37×10⁶ vs 1.45×10⁶ p=0.019) (FIG. 21). DCR-2-PBD significantly reduced the AML cells as a mean percentage of total cells compared to isotype-PBD (12.32% vs 38.92%, p=0.03).

### CONCLUSIONS

DCR-2-PBD demonstrated specific in vitro toxicity of AML cell lines and HSPCs. The PBD dimer component allows for rapid cytotoxicity and bystander killing.

PBD dimer, when internalized on an anti-CD300f antibody, causes significant cytotoxicity even with a brief exposure, which is ideal for a conditioning agent.

The even distribution across the major HSPC subtypes makes CD300f a more efficient conditioning agent compared with current AML targets being studied, which often have variable HSPC expression.

### SUMMARY

DCR-2-PBD exhibits the following properties:
likely has limited targets outside of the haematopoietic system
is readily internalised and able to deliver toxin
is potent against CD300f expressing AML cell lines in vitro and in vivo
depletes Healthy CD34+ haematopoietic progenitors
is effective within a 24-hour exposure
is able to kill mixed colonies of CD300f+ve and CD300f−ve cells
has a limited impact on antigen presentation
synergizes with fludarabine to enhance cytotoxicity.
Is effective for conditioning a subject for haematopoietic stem cell transplantation.

SEQUENCE LISTING

SEQ ID NO: 1
Met Glu Ser Gly Gly Gly Leu Val Gln Pro Gly Gly Pro Leu Lys Leu
Ser Cys Ala Ala Ser Gly Phe Gly Phe Ser Gly Ser Trp Met Ser Trp
Val Arg Gln Ala Pro Gly Lys Gly Leu Glu Trp Ile Gly Gln Ile Asn
Pro Asp Ser Ser Thr Ile Asn Tyr Thr Pro Ser Leu Lys Asp Lys Phe
Ile Ile Ser Arg Asp Asn Ala Lys Asn Thr Leu Tyr Leu Gln Ile Asn
Lys Val Arg Ser Glu Asp Thr Ala Leu Tyr Tyr Cys Ala Arg Arg Gly
Phe Phe Glu Gly Tyr Ser Ala Trp Phe Ala Tyr Trp
SEQ ID NO: 2
Gly Phe Gly Phe Ser Gly Ser Trp
SEQ ID NO: 3
Ile Asn Pro Asp Ser Ser Thr Ile
SEQ ID NO: 4
Ala Arg Arg Gly Phe Phe Glu Gly Tyr Ser Ala Trp Phe Ala Tyr
SEQ ID NO: 5
Ile Leu Met Thr Gln Thr Pro Lys Phe Leu Leu Val Ser Ala Gly Asp
Arg Val Thr Ile Thr Cys Lys Ala Ser Gln Ser Val Ser Asn Asp Val
Ala Trp Tyr Gln Gln Lys Pro Gly Gln Ser Pro Ser Leu Leu Ile Tyr
Tyr Ala Ser Asn Arg Asn Thr Gly Val Pro Asp Arg Phe Thr Gly Ser
Gly Tyr Glu Thr Asp Phe Thr Phe Thr Ile Ser Thr Val Gln Ala Glu
Asp Leu Ala Val Tyr Phe Cys Gln Gln Asp Tyr Thr Ser Pro Trp Thr
Phe Gly Gly Gly
SEQ ID NO: 6
Gln Ser Val Ser Asn Asp
SEQ ID NO: 7
Tyr Ala Ser
SEQ ID NO: 8
Gln Gln Asp Tyr Thr Ser Pro Trp Thr
SEQ ID NO: 9

| | |
|---|---|
| atggagtctg gaggtggcct ggtgcagcct ggaggacccc tgaaactctc ctgtgcagcc | 60 |
| tcaggattcg gttttagtgg atcttggatg agttgggtcc ggcaggctcc agggaaaggg | 120 |
| ctagaatgga ttggacaaat taatccagat agcagtacga taaattatac accatctcta | 180 |
| aaggataaat tcatcatctc cagagacaac gccaaaaata ccctgtacct gcaaattaac | 240 |
| aaagtgagat ctgaggacac agccctttat tactgtgcaa gacgggggtt ctttgaaggt | 300 |
| tactccgcct ggtttgctta ctgg | 324 |

SEQ ID NO: 10

| | |
|---|---|
| attttgatga cccagactcc caaattcctg cttgtatcag caggagacag ggtgaccata | 60 |
| acctgcaagg ccagtcagag tgtgagtaat gatgtagctt ggtaccaaca gaagccaggg | 120 |
| cagtctcctt cactcctgat atactatgca tccaatcgca acactggagt ccctgatcgc | 180 |
| ttcactggca gtggatatga gacggatttc actttcacca tcagcactgt gcaggctgaa | 240 |
| gacctggcag tttatttctg tcagcaggat tatacctctc cgtggacgtt cggtggaggc | 300 |

SEQ ID NO: 11

| | |
|---|---|
| atggtcctga gcctgctgta cctgctgaca gctctgcctg gcatcctgtc tgaggtccag | 60 |
| ctgcaagagt ctggccccat ggaatctggc ggaggattgg ttcaacctgg cggccctctg | 120 |
| aagctgtctt gtgccgcttc tggcttcggc ttctccggct cttggatgtc ctgggtccga | 180 |
| caggctcctg gcaaaggcct ggaatggatc ggccagatca accccgactc ctccaccatc | 240 |
| aactacaccc ctagcctgaa ggacaagttc atcatctccc gggacaacgc caagaacacc | 300 |
| ctgtacttgc agatcaacaa agtgcggagc gaggacaccg ctctgtacta ctgtgccaga | 360 |
| cggggcttct tcgagggcta ctctgcttgg tttgcctact ggggccaggg cacactggtc | 420 |
| acagtttctg ccgcctctac caagggaccc agcgttttcc ctctggctcc atcctccaag | 480 |
| tctacctctg gcggaacagc tgctctgggc tgcctggtca aggactactt tcctgagcca | 540 |
| gtgaccgtgt cctggaactc tggcgctctg acatctggcg tgcacacctt tccagctgtg | 600 |
| ctgcagtcct ccggcctgta ctctctgtcc tctgtcgtga ccgtgccttc cagctctctg | 660 |
| ggaacccaga cctacatctg caatgtgaac cacaagcctt ccaacaccaa ggtggacaag | 720 |
| aaggtggaac ccaagtcctg cgacaagacc cacacctgtc ctccatgtcc tgctccagaa | 780 |
| ctgctcggcg gaccttccgt gttcctgttt cctccaaagc ctaaggacac cctgatgatc | 840 |
| tctcggaccc ctgaagtgac ctgcgtggtg gtggatgtgt ctcacgagga tcccgaagtg | 900 |
| aagttcaatt ggtacgtgga cggcgtggaa gtgcacaatg ccaagaccaa gcctagagag | 960 |
| gaacagtaca actccaccta tagagtggtg tccgtgctga ccgtgctgca ccaggattgg | 1020 |
| ctgaacggca aagagtacaa gtgcaaggtg tccaacaagg ccctgcctgc tcctatcgaa | 1080 |
| aagaccatct ccaaggccaa gggccagcct agggaacccc aggtttacac cttgcctcca | 1140 |
| tctcgggacg agctgaccaa gaaccaggtg tccctgacct gtctcgtgaa gggcttctac | 1200 |
| ccctccgata tcgccgtgga atgggagtct aatggccagc ctgagaacaa ctacaagaca | 1260 |
| acccctcctg tgctggactc cgacggctca ttcttcctgt actccaagct gacagtggac | 1320 |
| aagtccagat ggcagcaggg caacgtgttc tcctgctccg tgatgcacga ggccctgcac | 1380 |
| aatcactaca cccagaagtc cctgtctctg tcccctggca ag | 1422 |

SEQ ID NO: 12

| | |
|---|---|
| atggactctc aggcccaggt gctgatgctg ctgctgttgt gggtgtccgg cacctgtggc | 60 |
| gacatcctga tgacccagac tcctaagttc ctgctggtgt ctgccggcga cagagtgacc | 120 |
| atcacatgca aggcctctca gtccgtgtcc aacgacgtgg cctggtatca gcagaagcct | 180 |
| ggccagtctc ctagcctgct gatctactac gcctccaaca gaaacaccgg cgtgcccgat | 240 |
| agattcaccg gctctggcta cgagacagac ttcaccttca ccatctccac cgtgcaggcc | 300 |
| gaggatctgg ccgtgtactt ctgccagcaa gactacacct ctccatggac ctttggcgga | 360 |
| ggcaccaagc tggaaatcaa gcggacagtg gccgctcctt ccgtgttcat cttcccacct | 420 |
| tccgacgagc agctgaagtc tggcacagcc tctgtcgtgt gcctgctgaa caacttctac | 480 |
| cctcgggaag ccaaggtgca gtggaaggtg gacaatgccc tgcagtccgg caactcccaa | 540 |
| gagtctgtga ccgagcagga ctccaaggac agcacctaca gcctgtcctc cacactgacc | 600 |
| ctgtccaagg ccgactacga gaagcacaag gtgtacgcct gcgaagtgac ccatcagggc | 660 |
| ctgtctagcc ctgtgaccaa gtctttcaac cggggcgagt gc | 702 |

SEQ ID NO: 13
Met Val Leu Ser Leu Leu Tyr Leu Leu Thr Ala Leu Pro Gly Ile Leu
Ser Glu Val Gln Leu Gln Glu Ser Gly Pro Met Glu Ser Gly Gly Gly
Leu Val Gln Pro Gly Gly Pro Leu Lys Leu Ser Cys Ala Ala Ser Gly
Phe Gly Phe Ser Gly Ser Trp Met Ser Trp Val Arg Gln Ala Pro Gly
Lys Gly Leu Glu Trp Ile Gly Gln Ile Asn Pro Asp Ser Ser Thr Ile
Asn Tyr Thr Pro Ser Leu Lys Asp Lys Phe Ile Ile Ser Arg Asp Asn
Ala Lys Asn Thr Leu Tyr Leu Gln Ile Asn Lys Val Arg Ser Glu Asp
Thr Ala Leu Tyr Tyr Cys Ala Arg Arg Gly Phe Phe Glu Gly Tyr Ser
Ala Trp Phe Ala Tyr Trp Gly Gln Gly Thr Leu Val Thr Val Ser Ala
Ala Ser Thr Lys Gly Pro Ser Val Phe Pro Leu Ala Pro Ser Ser Lys
Ser Thr Ser Gly Gly Thr Ala Ala Leu Gly Cys Leu Val Lys Asp Tyr
Phe Pro Glu Pro Val Thr Val Ser Trp Asn Ser Gly Ala Leu Thr Ser
Gly Val His Thr Phe Pro Ala Val Leu Gln Ser Ser Gly Leu Tyr Ser
Leu Ser Ser Val Val Thr Val Pro Ser Ser Ser Leu Gly Thr Gln Thr
Tyr Ile Cys Asn Val Asn His Lys Pro Ser Asn Thr Lys Val Asp Lys
Lys Val Glu Pro Lys Ser Cys Asp Lys Thr His Thr Cys Pro Pro Cys
Pro Ala Pro Glu Leu Leu Gly Gly Pro Ser Val Phe Leu Phe Pro Pro
Lys Pro Lys Asp Thr Leu Met Ile Ser Arg Thr Pro Glu Val Thr Cys
Val Val Val Asp Val Ser His Glu Asp Pro Glu Val Lys Phe Asn Trp
Tyr Val Asp Gly Val Glu Val His Asn Ala Lys Thr Lys Pro Arg Glu
Glu Gln Tyr Asn Ser Thr Tyr Arg Val Val Ser Val Leu Thr Val Leu
His Gln Asp Trp Leu Asn Gly Lys Glu Tyr Lys Cys Lys Val Ser Asn
Lys Ala Leu Pro Ala Pro Ile Glu Lys Thr Ile Ser Lys Ala Lys Gly
Gln Pro Arg Glu Pro Gln Val Tyr Thr Leu Pro Pro Ser Arg Asp Glu
Leu Thr Lys Asn Gln Val Ser Leu Thr Cys Leu Val Lys Gly Phe Tyr
Pro Ser Asp Ile Ala Val Glu Trp Glu Ser Asn Gly Gln Pro Glu Asn
Asn Tyr Lys Thr Thr Pro Pro Val Leu Asp Ser Asp Gly Ser Phe Phe
Leu Tyr Ser Lys Leu Thr Val Asp Lys Ser Arg Trp Gln Gln Gly Asn
Val Phe Ser Cys Ser Val Met His Glu Ala Leu His Asn His Tyr Thr
Gln Lys Ser Leu Ser Leu Ser Pro Gly Lys
SEQ ID NO: 14
Met Asp Ser Gln Ala Gln Val Leu Met Leu Leu Leu Leu Trp Val Ser
Gly Thr Cys Gly Asp Ile Leu Met Thr Gln Thr Pro Lys Phe Leu Leu
Val Ser Ala Gly Asp Arg Val Thr Ile Thr Cys Lys Ala Ser Gln Ser
Val Ser Asn Asp Val Ala Trp Tyr Gln Gln Lys Pro Gly Gln Ser Pro
Ser Leu Leu Ile Tyr Tyr Ala Ser Asn Arg Asn Thr Gly Val Pro Asp
Arg Phe Thr Gly Ser Gly Tyr Glu Thr Asp Phe Thr Phe Thr Ile Ser
Thr Val Gln Ala Glu Asp Leu Ala Val Tyr Phe Cys Gln Gln Asp Tyr
Thr Ser Pro Trp Thr Phe Gly Gly Gly Thr Lys Leu Glu Ile Lys Arg
Thr Val Ala Ala Pro Ser Val Phe Ile Phe Pro Pro Ser Asp Glu Gln
Leu Lys Ser Gly Thr Ala Ser Val Val Cys Leu Leu Asn Asn Phe Tyr
Pro Arg Glu Ala Lys Val Gln Trp Lys Val Asp Asn Ala Leu Gln Ser
Gly Asn Ser Gln Glu Ser Val Thr Glu Gln Asp Ser Lys Asp Ser Thr
Tyr Ser Leu Ser Ser Thr Leu Thr Leu Ser Lys Ala Asp Tyr Glu Lys
His Lys Val Tyr Ala Cys Glu Val Thr His Gln Gly Leu Ser Ser Pro
Val Thr Lys Ser Phe Asn Arg Gly Glu Cys
SEQ ID NO: 15
Met Glu Ser Gly Gly Gly Leu Val Gln Pro Gly Gly Ser Leu Arg Leu
Ser Cys Ala Ala Ser Gly Phe Gly Phe Ser Gly Ser Trp Met Ser Trp
Val Arg Gln Ala Pro Gly Lys Gly Leu Glu Trp Val Ala Asn Ile Asn
Pro Asp Ser Ser Thr Ile Asn Tyr Val Asp Ser Val Lys Gly Arg Phe
Thr Ile Ser Arg Asp Asn Ala Lys Asn Ser Leu Tyr Leu Gln Met Ser
Lys Val Arg Ser Glu Asp Thr Ala Leu Tyr Tyr Cys Ala Arg Arg Gly
Phe Phe Glu Gly Tyr Ser Ala Trp Phe Ala Tyr Trp

1. An immunoconjugate comprising an antibody, or antigen binding fragment thereof, linked to at least one pyrrolobenzodiazepine moiety, wherein the antibody, or antigen binding fragment thereof, specifically binds to an extracellular domain of CD300f and comprises:
(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

2. The immunoconjugate of claim 1, wherein the heavy chain variable region comprises a CDR1 comprising the amino acid sequence represented by SEQ ID NO: 2, a CDR2 comprising an amino acid sequence represented by SEQ ID NO: 3, and a CDR3 comprising the amino acid sequence represented by SEQ ID NO: 4.

3. The immunoconjugate of any claim 1, wherein the light chain variable region comprises a CDR1 comprising the amino acid sequence represented by SEQ ID NO: 6, a CDR2 comprising an amino acid sequence represented by SEQ ID NO: 7, and a CDR3 comprising the amino acid sequence represented by SEQ ID NO: 8.

4. The immunoconjugate of claim 1, wherein the heavy chain variable region comprises an amino acid sequence that is at least 70% identical to the amino acid sequence represented by SEQ ID NO: 1, and comprises a CDR1 comprising the amino acid sequence represented by SEQ ID NO: 2, a CDR2 comprising an amino acid sequence represented by SEQ ID NO: 3, and a CDR3 comprising the amino acid sequence represented by SEQ ID NO: 4.

5. The immunoconjugate of claim 1, wherein the light chain variable region comprises an amino acid sequence that is at least 70% identical to the amino acid sequence represented by SEQ ID NO: 5, and comprises a CDR1 comprising the amino acid sequence represented by SEQ ID NO: 6, a CDR2 comprising an amino acid sequence represented by SEQ ID NO: 7, and a CDR3 comprising the amino acid sequence represented by SEQ ID NO: 8.

6. The immunoconjugate of claim 1, wherein the heavy chain variable region comprises the amino acid sequence represented by SEQ ID NO: 1.

7. The immunoconjugate of claim 1, wherein the light chain variable region comprises the amino acid sequence represented by SEQ ID NO: 5.

8. The immunoconjugate of claim 1, wherein the heavy chain comprises the amino acid sequence represented by SEQ ID NO: 13.

9. The immunoconjugate of claim 1, wherein the light chain comprises the amino acid sequence represented by SEQ ID NO: 14.

10. The immunoconjugate of claim 1, wherein the pyrrolobenzodiazepine moiety is a pyrrolobenzodiazepine dimer.

11. The immunoconjugate of claim 10, wherein the pyrrolobenzodiazepine dimer is linked to the antibody, or antigen binding fragment thereof, via a linker.

12. The immunoconjugate of claim 11, wherein the linker comprises a peptide.

13. A pharmaceutical composition comprising the immunoconjugate of claim 1.

14. A method of treating a condition associated with CD300f expression, comprising administering an effective amount of the immunoconjugate of claim 1.

15. The method of claim 14, wherein the condition is AML.

16. (canceled)

17. A method of preparing a subject for haematopoietic stem cell transplantation, comprising administering to the subject:
an effective amount of an immunoconjugate comprising an antibody, or an antigen binding fragment thereof, linked to at least one cytotoxic agent, wherein the antibody, or antigen binding fragment thereof, specifically binds CD300f.

18. The method of claim 17, wherein the cytotoxic agent is at least one pyrrolobenzodiazepine moiety.

19. The method of claim 45, wherein the lymphodepleting agent is fludarabine or a pharmaceutically acceptable salt thereof.

20. The method of claim 45, wherein the cytotoxic agent is PBD dimer and the lymphodepleting agent is fludarabine or a pharmaceutically acceptable salt thereof.

21. The method of claim 17, wherein the antibody, or antigen binding fragment thereof, which specifically binds CD300f comprises:
(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

22. The method of claim 17, wherein the antibody, or antigen binding fragment thereof, which specifically binds CD300f comprises:
(a) a heavy chain variable region which comprises a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and a complementarity determining region 3 (CDR3) that comprises an amino acid sequence that is represented by SEQ ID NO: 4; and
(b) a light chain variable region which comprises a complementarity determining region 1 (CDR1) that comprises an amino acid sequence represented by SEQ ID NO: 6, a complementarity determining region 2 (CDR2) that comprises an amino acid sequence represented by SEQ ID NO: 7, and a complementarity determining region 3 (CDR3) that comprises an amino acid sequence represented by SEQ ID NO: 8.

23.-44. (canceled)

45. The method of claim 17, further comprising administering to the subject an effective amount of a lymphodepleting agent.

46. The method of claim 17, wherein the immunoconjugate comprises an antibody, or antigen binding fragment thereof, linked to at least one pyrrolobenzodiazepine moiety, wherein the antibody, or antigen binding fragment thereof, specifically binds to an extracellular domain of CD300f and comprises:
(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

47. A pharmaceutical composition for preparing a subject for haematopoietic stem cell transplantation, comprising:
(a) an immunoconjugate comprising an antibody, or an antigen binding fragment thereof, linked to at least one cytotoxic agent, wherein the antibody, or antigen binding fragment thereof, specifically binds CD300f; and
(b) a lymphodepleting agent.

48. The pharmaceutical composition of claim 47, wherein the cytotoxic agent is a pyrrolobenzodiazepine moiety.

49. The pharmaceutical composition of claim 48, wherein the pyrrolobenzodiazepine moiety is a pyrrolobenzodiazepine dimer.

50. The pharmaceutical composition of claim 47, wherein the immunoconjugate comprises an antibody, or antigen binding fragment thereof, linked to at least one pyrrolobenzodiazepine moiety, wherein the antibody, or antigen binding fragment thereof, specifically binds to an extracellular domain of CD300f and comprises:
(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

51. The pharmaceutical composition of claim 47, wherein the lymphodepleting agent is fludarabine or a pharmaceutically acceptable salt thereof.

52. A kit for preparing a subject for haematopoietic stem cell transplantation, comprising:
(a) an immunoconjugate comprising an antibody, or an antigen binding fragment thereof, linked to at least one cytotoxic agent, wherein the antibody, or antigen binding fragment thereof, specifically binds CD300f; and
(b) a lymphodepleting agent.

53. The kit of claim 52, wherein the cytotoxic agent is a pyrrolobenzodiazepine moiety.

54. The kit of claim 53, wherein the pyrrolobenzodiazepine moiety is a pyrrolobenzodiazepine dimer.

55. The kit of claim 52, wherein the immunoconjugate comprises an antibody, or antigen binding fragment thereof, linked to at least one pyrrolobenzodiazepine moiety, wherein the antibody, or antigen binding fragment thereof, specifically binds to an extracellular domain of CD300f and comprises:
(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.

56. The kit of claim 52, wherein the lymphodepleting agent is fludarabine or a pharmaceutically acceptable salt thereof.

57. A method of depleting haematopoietic stem and progenitor cells in a subject, comprising administering to the subject an effective amount of an immunoconjugate comprising an antibody, or an antigen binding fragment thereof, linked to at least one cytotoxic agent, wherein the antibody, or antigen binding fragment thereof, specifically binds CD300f.

58. The method of claim 57, wherein the cytotoxic agent is a pyrrolobenzodiazepine moiety.

59. The method of claim 58, wherein the pyrrolobenzodiazepine moiety is a pyrrolobenzodiazepine dimer.

60. The method of claim 57, wherein the immunoconjugate comprises an antibody, or antigen binding fragment thereof, linked to at least one pyrrolobenzodiazepine moiety, wherein the antibody, or antigen binding fragment thereof, specifically binds to an extracellular domain of CD300f and comprises:
(a) a heavy chain variable region which comprises:
(i) an amino acid sequence that is at least 70% identical to the amino acid sequence of the amino acid sequence represented by SEQ ID NO: 1; and/or
(ii) a complementarity determining region 1 (CDR1) that comprises the amino acid sequence represented by SEQ ID NO: 2, a complementarity determining region 2 (CDR2) that comprises an amino acid sequence that is represented by SEQ ID NO: 3, and/or a complementarity determining region 3 (CDR3) that comprises an amino acid sequence that is represented by SEQ ID NO: 4; and
(b) a light chain variable region which comprises:
(i) an amino acid sequence that is at least 70% identical to the amino acid sequence represented by SEQ ID NO: 5; and/or
(ii) a complementarity determining region 1 (CDR1) that comprises an amino acid sequence represented by SEQ ID NO: 6, a complementarity determining region 2 (CDR2) that comprises an amino acid sequence represented by SEQ ID NO: 7, and/or a complementarity determining region 3 (CDR3) that comprises an amino acid sequence represented by SEQ ID NO: 8.
